SEQUENCE LISTING (4 sequences)

SEQ ID NO: 1 - DNA, 22 bp, synthetic construct
  misc_feature 1..22: note = Single strand DNA oligonucleotide
  source 1..22: mol_type = other DNA
ggtgttcaaggtctcccacaag

SEQ ID NO: 2 - DNA, 22 bp, synthetic construct
  misc_feature 1..22: note = Single strand DNA oligonucleotide
  source 1..22: mol_type = other DNA
ccacgatgtacggagagttgca

SEQ ID NO: 3 - DNA, 20 bp, synthetic construct
  misc_feature 1..20: note = Single strand DNA oligonucleotide
  source 1..20: mol_type = other DNA
ccatcgcaaaccgctgccat

SEQ ID NO: 4 - DNA, 22 bp, synthetic construct
  misc_feature 1..22: note = Single strand DNA oligonucleotide
  source 1..22: mol_type = other DNA
aacacttctcagctatgggctt

(19) United States
(12) Patent Application Publication
(10) Pub. No.: US 2023/0273211 A1
(43) Pub. Date: Aug. 31, 2023

(54) METHOD OF DIAGNOSING BREAST CANCER

(71) Applicants: Yeda Research and Development Co. Ltd., Rehovot (IL); Tel HaShomer Medical Research Infrastructure and Services Ltd., Ramat-Gan (IL)

(72) Inventors: Sima LEV, Ramat-Efal (IL); Yaron VINIK, Rehovot (IL); Francisco Gabriel ORTEGA, Rehovot (IL); Mordechai GUTMAN MD, Tel-Aviv (IL)

(73) Assignees: Yeda Research and Development Co. Ltd., Rehovot (IL); Tel HaShomer Medical Research Infrastructure and Services Ltd., Ramat-Gan (IL)

(21) Appl. No.: 18/128,260

(22) Filed: Mar. 30, 2023

Related U.S. Application Data
(63) Continuation of application No. PCT/IL2021/051182, filed on Sep. 30, 2021.

(30) Foreign Application Priority Data
Oct. 1, 2020 (IL) 277743

(51) Int. Cl. G01N 33/574; G01N 1/34

(52) U.S. Cl. CPC G01N 33/57415; G01N 1/34; G01N 33/57488; G01N 2333/912; G01N 2333/4703; G01N 2333/78; G01N 2800/56; G01N 2800/52

(57) ABSTRACT

A method of diagnosing breast cancer is disclosed. The method comprises lysing extracellular vesicles of a subject to generate a composition comprising components of extracellular vesicles; measuring the amount of MEK1 in the composition; and diagnosing the subject with breast cancer when a level of said MEK1 in said composition is above a predetermined threshold. Kits for breast cancer diagnosis are also disclosed.

### RELATED APPLICATIONS

This application is a Continuation of PCT Patent Application No. PCT/IL2021/051182 having International filing date of Sep. 30, 2021, which claims the benefit of priority of Israeli Patent Application No. 277743 filed on Oct. 1, 2020. The contents of the above applications are all incorporated by reference as if fully set forth herein in their entirety.

### SEQUENCE LISTING STATEMENT

The XML file, entitled 95757SequenceListing.xml, created on Mar. 5, 2023, comprising 5,934 bytes, submitted concurrently with the filing of this application is incorporated herein by reference.

### FIELD AND BACKGROUND OF THE INVENTION

The present invention, in some embodiments thereof, relates to methods of diagnosing cancer, but not exclusively, to breast cancer.

Early detection of breast cancer (BC) has an important clinical impact on cancer therapy and overall survival. Currently, mammography, ultrasound, and MRI are commonly used for screening, but these methods are not always reliable, safe and/or cost effective. As a complement to imaging approaches, proteomic profiling of circulating extracellular vesicles (EVs) derived from plasma of BC patients represents a promising approach for early detection, diagnosis and prognosis (2). Increasing evidence suggest that protein contents of small EVs of 30-100 nm in size, such as exosomes or exosome-like vesicles (ELVs), can be used for assessing tumor prognosis and therapeutic responses. EVs proteins are more stable than other serological proteins as they are protected from circulating proteases by a lipid bilayer and thus could be better markers.

Unlike apoptotic blebs (50-5000 nm) that are released from apoptotic cells, EVs (50-1000 nm diameter) are released from multiple cell types including leukocytes, platelets, fibroblasts, adipocytes and cancer cells. Small extracellular vesicles (sEVs) of ˜100 nm diameter are generated from different subcellular compartments including the plasma membrane and multivesicular bodies (MVBs) and can be found in diverse body fluids such as semen, urine, saliva, breast milk, aminiotic fluid, cerebrospinal fluid and blood. sEVs have unique morphology and density, and thus, can be isolated by differential centrifugation and identified by electron microscopy (EM). In addition, sEVs contain a restricted set of proteins, miRNA, mRNA and DNA, and play important roles in cell-cell communication by transferring their content to target cells. sEVs are robustly produced by cancer cells and markedly affect the primary tumor microenvironment (TME) including the immune ecosystem as well as distant metastatic niches, thereby facilitating tumor growth and metastasis.

Tumor biopsies are currently considered as “gold standard” of diagnosis, prognosis and prediction of therapeutic response. In metastatic patients, tumor biopsy is limited by sampling a single metastatic site amongst many present, and in terms of longitudinal analysis, it is associated with potential morbidity and patient inconvenience. sEVs, in contrast, may provide unique information about the full metastatic complement of tumors and allow facile longitudinal analysis of tumor evolution in response to therapy.

Background art includes Galindo-Hernandez, O., et al., (2013) Arch Med Res 44, 208-214; Moon, P. G., et al., (2016) Oncotarget 7, 40189-40199; Meng et al., 2019, Technology in Cancer Research & Treatment Volume 18: 1-14; Huang et al., International Journal of Biological Sciences 2019; 15(1): 1-11.

### SUMMARY OF THE INVENTION

According to an aspect of the present invention there is provided a method of diagnosing breast cancer in a subject, comprising:

(a) isolating extracellular vesicles from serum and/or plasma of the subject to generate an isolated population of extracellular vesicles;

(b) lysing the isolated population of extracellular vesicles to generate a composition comprising components of the extracellular vesicles;

(c) measuring the amount of MEK1 in the composition; and

(d) diagnosing the subject with breast cancer when a level of the MEK1 in the composition is above a predetermined threshold.

According to an aspect of the present invention there is provided a method of diagnosing breast cancer in a subject, comprising:
(a) isolating extracellular vesicles from serum and/or plasma of the subject to generate an isolated population of extracellular vesicles;
(b) lysing the population of extracellular vesicles to generate a composition comprising components of the extracellular vesicles;
(c) measuring the amount of each of the proteins MEK1, fibronectin, FAK, β-Actin, C-Raf, N-Cadherin and P90RSK_pT573 in the composition; and
(d) diagnosing the subject with breast cancer based on the amount of each of the proteins.

According to an aspect of the present invention there is provided a kit for diagnosing breast cancer, the kit comprising an antibody that specifically binds to fibronectin, an antibody that specifically binds to FAK and an antibody that specifically binds to MEK1, wherein the number of target proteins for the antibodies of the kit is no greater than 20. According to an aspect of the present invention there is provided a method of treating breast cancer of a subject in need thereof, the method comprising:
(a) diagnosing the breast cancer according to any one of claims 1-10; and upon confirmation of breast cancer in the subject
(b) administering to the subject a therapeutic agent which is directed against breast cancer cells of the subject.

According to an aspect of the present invention there is provided a method of staging breast cancer in a subject in need thereof, comprising:
(a) isolating extracellular vesicles from serum and/or plasma of the subject to generate an isolated population of extracellular vesicles;
(b) lysing the extracellular vesicles to generate a composition comprising isolated components of the extracellular vesicles;
(c) measuring the amount of at least one protein selected from the group consisting of P-Cadherin, TAZ, cleaved caspase-7, EGFR, E2F1, Aurora-B, IGFRβ, NF-κB -p65 in the composition; and
(d) staging the breast cancer according to the amount.

According to an aspect of the present invention there is provided a method of determining the risk of breast cancer relapse in a subject, comprising:
(a) isolating extracellular vesicles from serum and/or plasma of the subject to generate an isolated population of extracellular vesicles;
(b) lysing the extracellular vesicles to generate a composition comprising components of the extracellular vesicles;
(c) measuring an expression level of at least one of the proteins selected from the group consisting of MIF, COGS, Cox-IV, cyclophilin-F, EMA, HSP70, MMP2 and VEGFR-2 in the composition, wherein the expression level of the at least one of the proteins correlates with the risk of breast cancer relapse.

According to embodiments of the present invention, the composition is a protein extract.

According to embodiments of the present invention, the method further comprises measuring the amount of fibronectin and FAK in the composition, wherein a level of the MEK1, the fibronectin and the FAK in the composition above a predetermined threshold is indicative that the subject has breast cancer.

According to embodiments of the present invention, the isolated population of extracellular vesicles are between 30-150 nM in diameter.

According to embodiments of the present invention, the method further comprises measuring the amount of the proteins β-Actin, C-Raf, N-Cadherin and P90RSK_pT573 in the composition, wherein a level of the proteins below a predetermined threshold in the composition is indicative that the subject has breast cancer.

According to embodiments of the present invention, the isolating comprises purifying the extracellular vesicles from serum albumin.

According to embodiments of the present invention, the purifying is effected by performing size exclusion chromatography and/or filtration on the isolated population of extracellular vesicles.

According to embodiments of the present invention, the purifying is effected by depleting the composition of serum albumin.

According to embodiments of the present invention, the isolated population of extracellular vesicles comprise exosomes.

According to embodiments of the present invention, the diagnosing is effected using a machine learning algorithm.

According to embodiments of the present invention, the measuring comprises measuring the amount of each of the proteins P-Cadherin, TAZ, cleaved caspase-7, EGFR, E2F1, Aurora-B, IGFRβ, NF-κB-p65 in the composition.

According to embodiments of the present invention, the staging is effected using a machine learning algorithm.

According to embodiments of the present invention, the composition comprises a protein extract.

According to embodiments of the present invention, the isolating comprises purifying the extracellular vesicles from serum albumin.

According to embodiments of the present invention, the isolated population of extracellular vesicles are between 30-150 nM in diameter.

According to embodiments of the present invention, a level of the P-Cadherin, TAZ and/or cleaved caspase-7 below a predetermined threshold is indicative that the subject has breast cancer at a stage later than Stage 1.

According to embodiments of the present invention, a level of the EGFR, E2F1, Aurora-B below a predetermined threshold is indicative that the subject has Stage I breast cancer.

According to embodiments of the present invention, the method comprises analyzing IGFRβ and NF-κB-p65, wherein a level of both the IGFRβ and the NF-κB-p65 below a predetermined threshold is indicative that the subject has a cancer later than stage I.

According to embodiments of the present invention, the subject has stage I or stage IIA cancer.

According to embodiments of the present invention, the subject has been diagnosed with breast cancer according to the method disclosed herein.

According to embodiments of the present invention, the method further comprises analyzing the size distribution of extracellular vesicles derived from the serum and/or plasma of the subject, wherein the lower the size distribution of the extracellular vesicles, the later the stage of the breast cancer.

According to embodiments of the present invention, the extracellular vesicles comprise exosomes.

According to embodiments of the present invention, the method comprises treating the subject with an agent appropriate for the stage of the breast cancer.

According to embodiments of the present invention, the composition comprises a protein extract.

According to embodiments of the present invention, the extracellular vesicles of the isolated population are between 30-150 nM in diameter.

According to embodiments of the present invention, the isolating comprises purifying the extracellular vesicles from serum albumin.

According to embodiments of the present invention, the level of MIF, COG3, Cox-IV, cyclophilin-F, EMA or HSP70 is above a predetermined threshold, it is indicative of a high risk for breast cancer relapse.

According to embodiments of the present invention, the level of MMP2 and VEGFR-2 is below a predetermined threshold, it is indicative of a high risk of breast cancer relapse.

According to embodiments of the present invention, the method further comprises analyzing the Oncotype recurrence score (RS) of the subject.

According to embodiments of the present invention, the extracellular vesicles of the isolated population comprise exosomes.

According to embodiments of the present invention, the number of target proteins for the antibodies of the kit is no greater than 10.

According to embodiments of the present invention, the kit further comprises an antibody that specifically binds to β-Actin, an antibody that specifically binds to C-Raf, an antibody that specifically binds to N-Cadherin and an antibody that specifically binds to P90RSK_pT573.

According to embodiments of the present invention, the at least one of the antibody is a monoclonal antibody.

According to embodiments of the present invention, the least one of the antibody is attached to a solid support.

According to embodiments of the present invention, each of the antibodies is attached to a solid support.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E: sEVs extraction from human plasma samples.

FIG. 1A—A scheme depicting the procedure for EVs enrichment and extraction. sEVs were partially purified from the plasma of patients by serial centrifugations, filtration and passing through size exclusion chromatography (SEC). Fractions of 1.5 ml were collected from the SEC eluent.

FIG. 1B—Size distribution of particles in the different fractions as measured by NanoSight. Shown is a representative chart of three independent repeats.

FIG. 1C—sEVs markers in the different SEC fractions. Western Blot (WB) analysis of protein extracts of the indicated fractions from 2 plasma samples, as a representative of at least 4 samples.

FIG. 1D—Commassie Brilliant blue staining of protein extracted from the different fractions and the original plasma (diluted 1:40). Shown is a representative of two repeats. Similar volume of indicated fraction was loaded.

FIG. 1E—Fractions purity was calculated as a log ratio between particles number to protein concentration (n=6 plasma samples), and significant differences between fraction 3 and 4 were determined by t-test.

FIGS. 2A-J: RPPA analysis of sEVs-enriched fractions (A-I) Plasma EVs extracted from pre-surgery BC patients (n=52) and healthy controls (n=22) were analyzed by RPPA.

FIG. 2A—Volcano plot showing differentially expressed proteins between pre-surgery patients and healthy controls. The top hits are marked in red (upregulated proteins) or blue (downregulated proteins).

FIG. 2B—Principal component analysis (PCA) of the BC patients and healthy control using expression levels of the 60 top significantly different proteins (yielding the maximum partition in the cohort).

FIG. 2C—Unsupervised clustering of the entire cohort using the 10 proteins selected by the kNN test. Each row indicates one woman, either healthy control (orange) or pre-surgery patients (red).

FIG. 2D—Logistic regression with elastic net penalty performed on the main cohort. Shown is the importance plot of the proteins in the model (based on their z-statistic, and normalized on a scale from 0 to 100). Arrows in the bars indicate the proteins that appear in the kNN signature and are up- or down-regulated in BC vs. healthy.

FIG. 2E—Unsupervised clustering of the entire cohort using the 7 proteins selected both by the kNN test and the logistic regression model.

FIG. 2F—Accuracy parameters of the clustering in FIGS. 1C and E.

FIG. 2G—ROC curves of the 3 upregulated proteins in the signature.

FIG. 2H—Boxplot depicting the expression and distribution of the 3 upregulated proteins in the signature.

FIG. 2I—Pairwise similarity matrix based on Spearman's correlations of 276 protein in the BC patients, clustered into 8 partitions. ‘1’ and ‘2’ indicate the partitions that includes the 3 upregulated proteins and 3 of the 4 downregulated proteins from E, respectively.

FIG. 2J—RPPA validation by Western blotting. Shown are representative Western blots of the 3 upregulated proteins of the signature. Densitometry results of at least 4 healthy and 4 patients are shown in the right panels.

FIGS. 3A-C: Validation of the 7-protein signature.

FIGS. 3A-C—Validation of the results on an independent test set. Plasma EVs were extracted from 16 BC patients (blood taken during surgery) and 8 healthy controls, and were analyzed by RPPA.

FIG. 3A—clustering of the test set samples using the 7-proteins signature obtained using kNN and logistic regression on the main cohort.

FIG. 3B—ROC curves and AUC values of several proteins from the signature, done on the test set samples.

FIG. 3C—Machine learning models used to classify the test set samples. All models were performed using 268 proteins appearing both in the main cohort and the test set RPPA. Models were trained on the main cohort to tune model parameters. Models were applied on the test set samples, and confusion matrixes were built to calculate accuracy, sensitivity, specificity and positive and negative predictive values (PPV, NPV).

FIGS. 4A-D: Effects of breast cancer stage on number and protein content of EVs.

FIG. 4A-C—Light scattering (NanoSight) was used to generate a size histogram of the EVs in the enriched plasma fractions from healthy women or stage I or stage IIA patients.

FIG. 4A—Histogram shown is the average of n=20 healthy controls, 12 stage I and 6 stage IIA patients. The number of low-size sEVs (smaller than 100 nM) was quantified in (B).

FIG. 4C-D—kNN tests were used to generate protein signature to classify stage I and stage IIA patients. Unsupervised clustering of pre-surgery stage I patients (C, left) or stage IIA patients (D, left) (in red) with the healthy controls (in orange) are shown using the generated signature. Logistic regression with elastic net penalty was built for each classification, and variables importance plot of the variables in the model (based on their z-statistic, and normalized on a scale from 0 to 100) are shown on the right. Arrows in the bars indicate the proteins that appear in the kNN signature and are up- or down-regulated in the relevant signature (n=25 stage I patients, 11 stage IIA patients, 22 healthy controls).

FIGS. 5A-C: Relapse prediction and associated proteins.

FIG. 5A—Partition clustering of the breast cancer patients in the study. Clustering was done by the k-means method. Partitions are shown in PCA plot using the two highest principal components. Colors distinguish between 6 partitions (3 of them include single points). Red points represent the patients that underwent relapse. Numbers below some of the points is the Oncotype recurrence score (RS) for those patients.

FIG. 5B—Oncotype RS s were measured for 16 of the patients. Red bars mark the patients that underwent relapse.

FIG. 5C—Volcano plot showing differentially expressed proteins between the 3 patients that underwent relapse plus patient number 15 from the Oncotype RS=31 versus the other patients.

FIGS. 6A-E: Analysis of post-surgery samples.

FIGS. 6A-B—Plasma samples from 27 patients were collected ˜24 weeks after surgery. Particle distribution (A) and number of sEVs smaller than 100 nm diameter (B) was measured in 23 samples of the post-surgery by NanoSight analysis.

FIG. 6C—Unsupervised clustering of the post-surgery samples and the healthy controls. The close up of the dendrogram zoom in on the post-surgery samples cluster, detailing the adjuvant chemotherapy regiment for the patients that received it before the plasma sample was taken. Gray indicates healthy controls (n=22), orange are post-surgery samples that received adjuvant chemotherapy (n=8) and red are post-surgery samples that did not (n=19).

FIG. 6D—PCA analysis of post-surgery samples using the significant differently expressed proteins (p-value<0.05) between samples after chemotherapy and samples without chemotherapy.

FIG. 6E—Volcano plot showing the significant differently expressed proteins in patients undergoing chemotherapy versus non.

FIGS. 7A-G: Selected biomarker for breast cancer patients.

FIG. 7A—K-nearest neighbor (kNN) models were trained using the leave-one-out cross validation method for N=1 to 276 top significant proteins differently expressed in breast cancer (BC) patients pre-surgery versus healthy controls, based on the closest neighbor (k=1). ROC-AUC was determined for each model. Clustering based on the maximum point at N=60 is given in FIG. 2B (n=52 patients and 22 healthy controls).

FIG. 7B—kNN models for different combinations of k (number of neighbors) and N (number of top significant features). Clustering based on the local maximum point at N=10, k=11 is given in FIG. 2C (n=52 patients and 22 healthy controls).

FIG. 7C—Logistic regression with elastic net penalty was trained on the main cohort. The black dot marks the model with the best accuracy (determined by 10-fold cross validation), which was used for the model in FIG. 2D.

FIG. 7D—Scatter plot showing correlation (pearson) between FAK and markers in the breast cancer cohort (n=7-8 patients for whom serum antigens were measured).

FIG. 7E—Decision tree model built on the breast cancer cohort. Each terminal node shows the predicted class (BC or healthy) as well as the probability of being healthy, and the percentage of samples within that node.

FIG. 7F—ROC curve for the downregulated proteins in the BC versus healthy controls signature.

FIG. 7G—Boxplot of expression levels of proteins from FIG. 7F.

FIGS. 8A-C: Validation of the 7-protein signature.

FIG. 8A—PCA plot of all samples in the main cohort and the test set.

FIG. 8B—Partition clustering of the test set samples using the 7-protein signature.

FIG. 8C—Boxplot of protein expression levels of the test set samples related to the ROC curve of FIG. 3B.

FIGS. 9A-F: sEVs number in subsets of BC with different features.

FIGS. 9A-B—Distribution of the % of infected nodes (A) and tumor size (B) among cancer stages.

FIG. 9C—Electron microscopy images of sEVs-enriched fractions extracted from plasma of healthy control and stage I and IIA patients. Scale bar=100 nm.

FIG. 9D—Total particle concentrations in healthy and stage I and IIA patients, calculated by light scattering based on the histograms of FIG. 4A.

FIG. 9E—sEVs concentration in different BMI categories of BC patients.

FIG. 9F—ROC curve for the number of sEVs smaller than 100 nm as a parameter distinguishing stage IIA patients from healthy controls.

FIGS. 10A-H: Selected biomarkers for cancer stage.

FIG. 10A—kNN models for classification of BC stage I versus healthy, stage IIA versus healthy and stage IIA versus stage I, using different k (number of neighbors) and N (number of top significant proteins). AUC was used as performance metric of each model. In each panel the model with the highest AUC is marked by a black point.

FIG. 10B—Protein signatures between all patients, only stage I or only stage IIA versus the healthy controls. Numbers indicate the fold change (log 2) between the patients in that group and the healthy controls.

FIG. 10C—Venn diagram showing the common and unique proteins in the signatures from FIG. 10B.

FIG. 10D—ROC curves for the best biomarkers distinguishing between stage I patients and healthy controls.

FIG. 10E—ROC curves for the best biomarkers distinguishing between stage IIA patients and healthy controls.

FIG. 10F—ROC curves for the best biomarkers distinguishing between stage I and stage IIA cancer patients.

FIG. 10G—Correlation (Pearson) between protein expression and number of small size (<100 nm) sEVs in the breast cancer patients (n=15 for SRC pY419, 28 for the rest).

FIG. 10H—Boxplots show the expression levels of all markers used in the ROC curve analysis of FIGS. 10A-H.

FIGS. 11A-C: Selected biomarkers for cancer subtype.

FIGS. 11A-C—ROC curves and the related boxplots for the best biomarkers that differentiate between ER positive and negative (A), PR positive and negative (B), and HER2 positive and negative (C).

FIGS. 12A-B. Selected biomarkers for cancer relapse.

FIG. 12A—The 21 genes of the oncotype RS (Recurrence Score) signature.

FIG. 12B—Correlation between the expression of the indicated proteins in breast cancer patients to Oncotype RS scores (n=11-12).

FIGS. 13A-C. Analysis of plasma samples post-surgery.

FIG. 13A—Number of low size (<100 nm) sEVs in plasma samples versus the number of weeks following surgery in which each sample was taken.

FIG. 13B—Volcano plot shows the differentially expressed proteins in radiotherapy-treated patients versus non-radiotherapy treated patients.

FIG. 13C—Venn diagram showing the proteins that went down or up in both pre-surgery and post-surgery samples.

### DESCRIPTION OF SPECIFIC EMBODIMENTS OF THE INVENTION

The present invention, in some embodiments thereof, relates to methods of diagnosing cancer and, more particularly, but not exclusively, to breast cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Identification of biomarkers with sufficient sensitivity and specificity for early detection of breast cancer (BC) remains a major challenge. The present inventors now describe a simple and reliable method to isolate extracellular vesicles (EVs) from plasma of BC patients and analyze their proteome by a semi-quantitative method. The results described herein show that this approach could have a powerful diagnostic impact for early detection and prediction of recurrence risk.

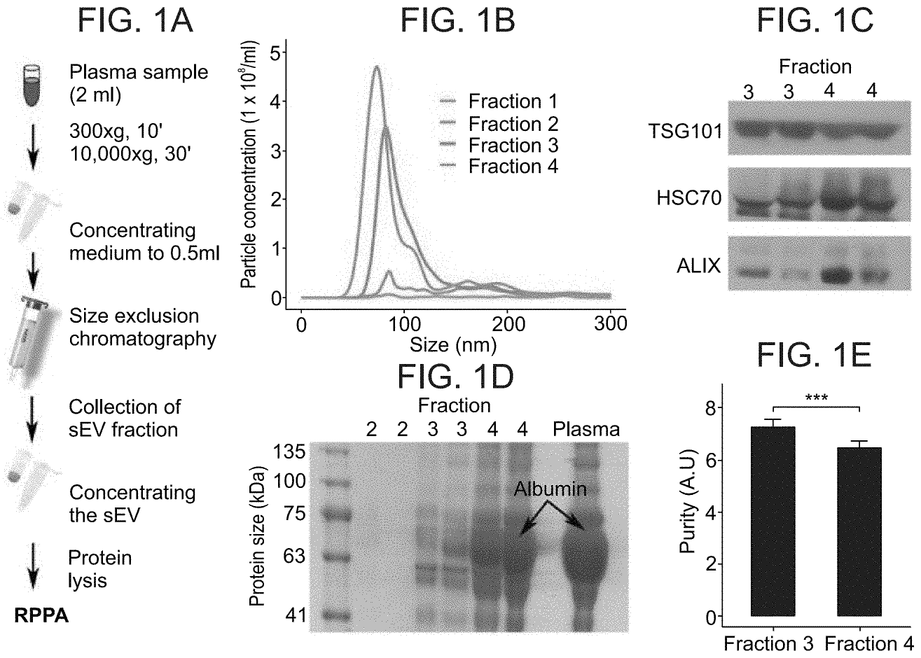

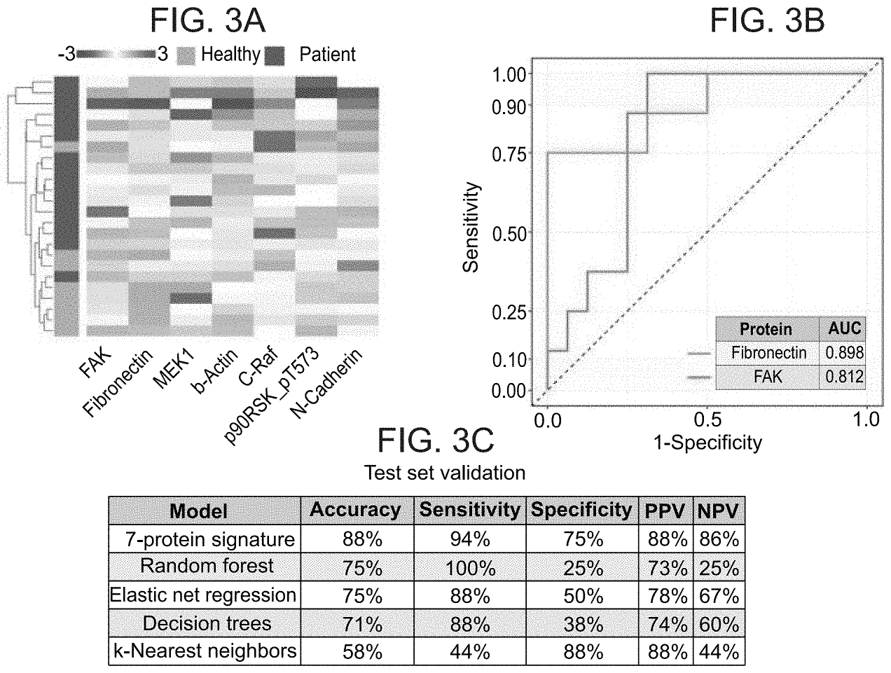

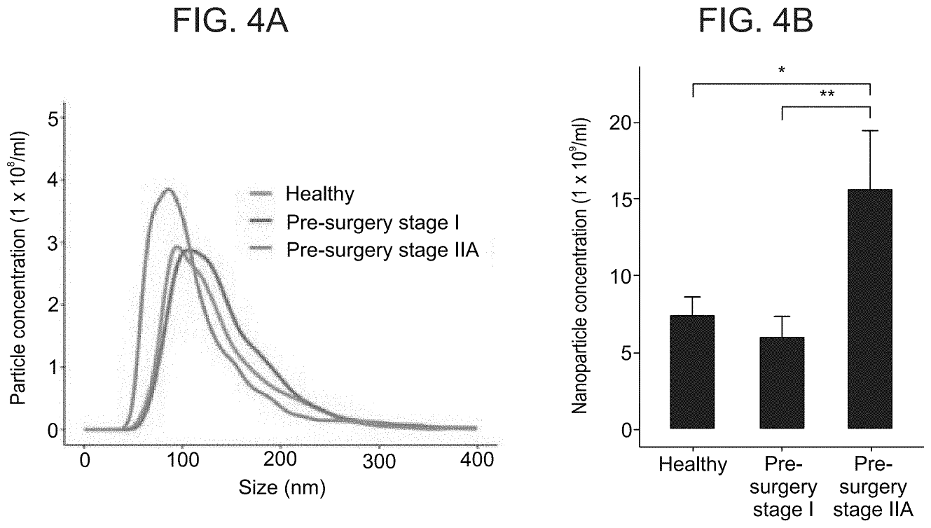

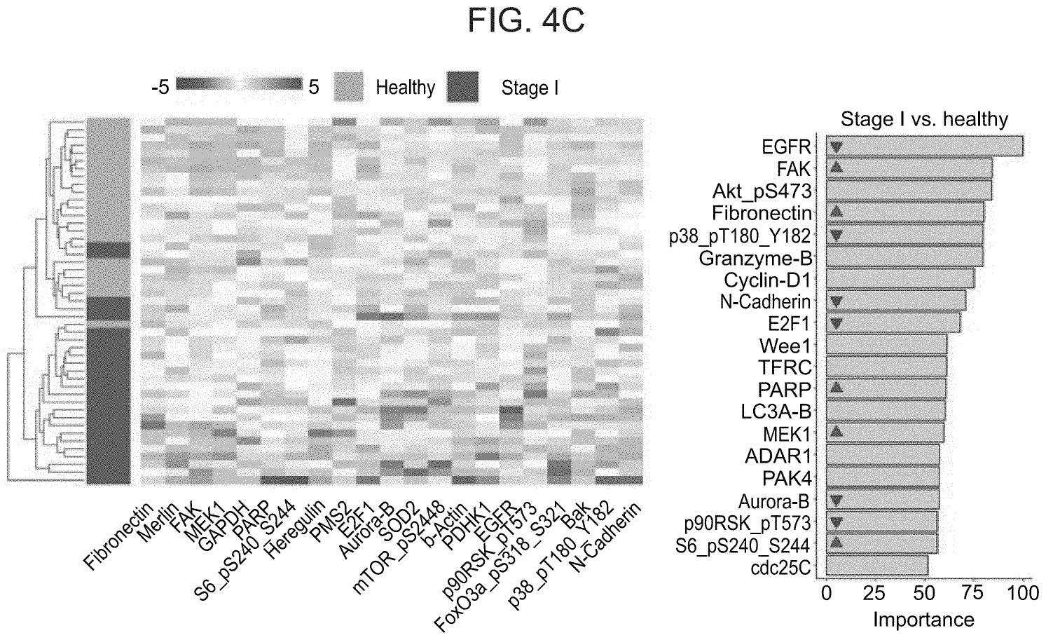

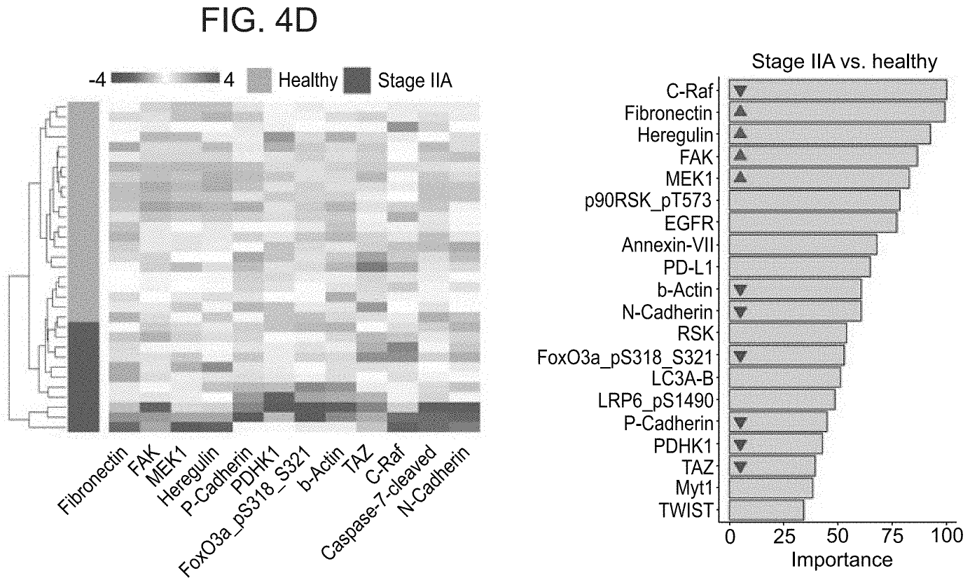

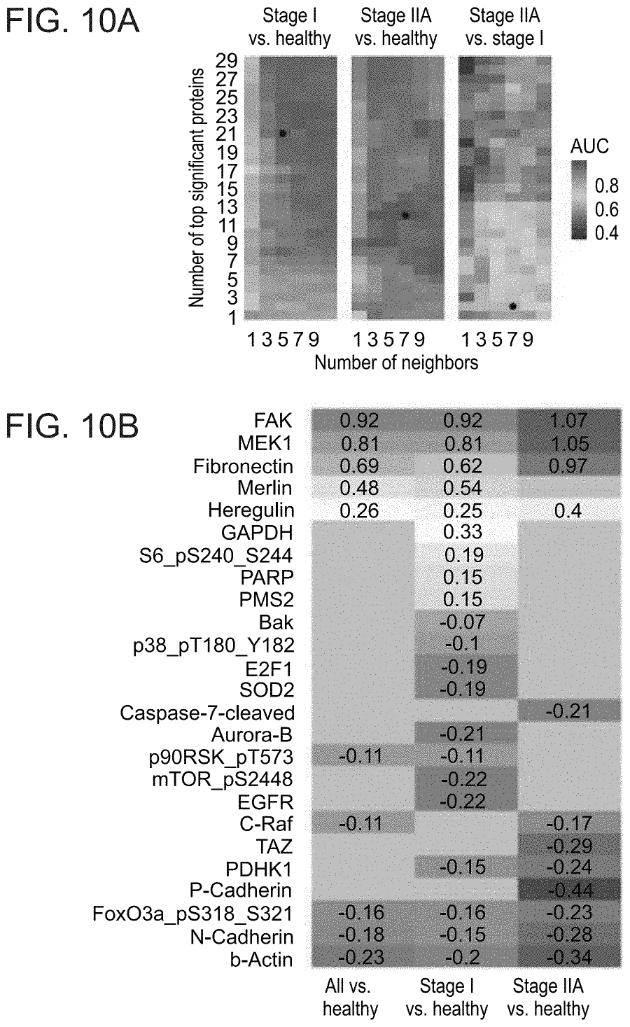

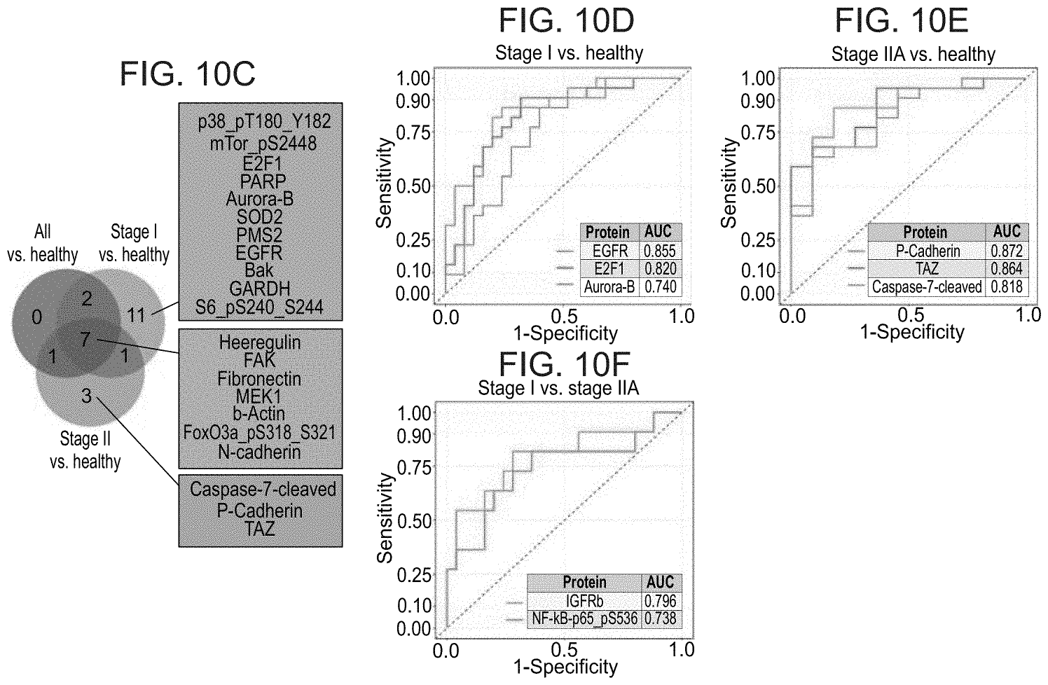

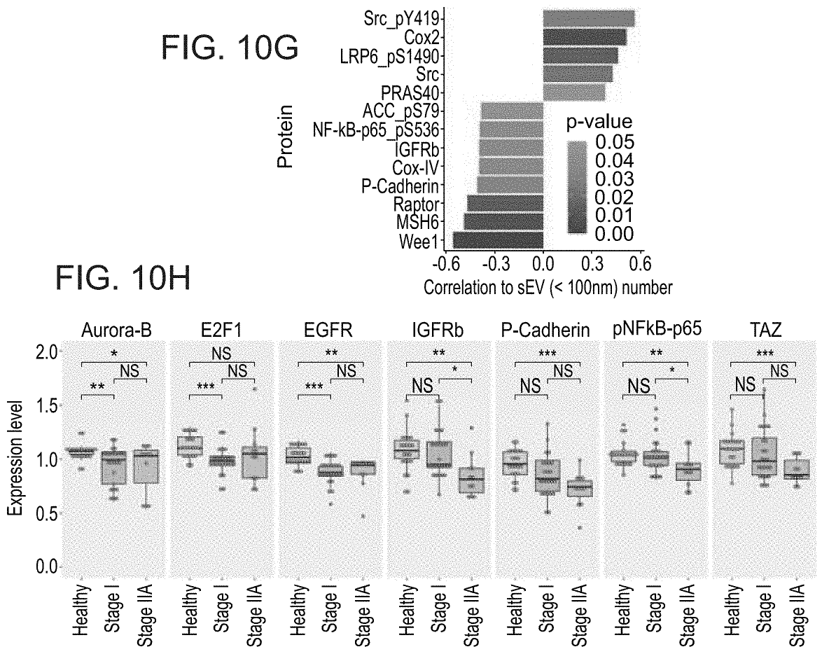

The present inventors established a simple protocol (FIG. 1A) to enrich small EVs (sEVs) of ˜100 nm in size, likely encompassing exosomes and exosome-like vesicles among other EVs (FIG. 1A, 1C), from plasma of BC patients. Analysis of expression profiles of ˜276 cancer-related proteins identified a signature of 7 proteins that clusters BC patients distinctly from healthy women in high accuracy, and thus could have important clinical impact. Validation of the 7-protein signature (FIGS. 3A-C) on an independent test set, taken from a different source, further strengthened these findings. The 7-protein signature yielded high accuracy of 88%, concomitant with a remarkable high sensitivity of 94%. Further analysis revealed that EVs can also be used to distinguish between stage I and stage IIA patients. Although the most profound difference was the increased number of smaller EVs of <100 nm (FIGS. 4A, B), the present inventors could define signatures and markers specific for these two stages. The most significantly differentially expressed proteins in stage IIA versus healthy women were P-Cadherin and TAZ (FIG. 10E), while IGFRβ was the best marker to differentiate between stage I and IIA (FIG. 10F). The levels of TAZ and P-Cadherin were reduced in stage IIA compared to healthy controls (FIG. 10H), and also have a negative correlation with numbers of small-sized sEVs as expected (FIG. 10G).

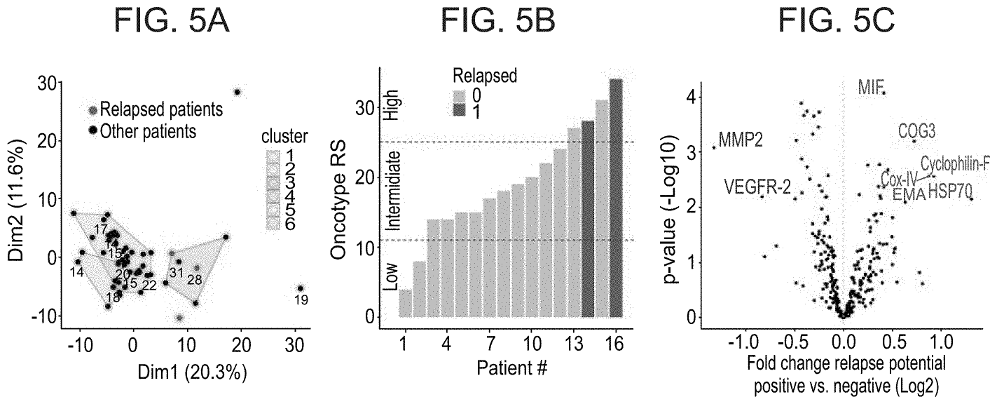

Prediction of relapse based on the cargo of early-stage sEVs could provide highly important information to guide treatment planning and consideration of possible outcomes. The present inventors found that the three relapsed patients in their cohort partitioned differently compared to most patients in the cohort (FIG. 5A). Furthermore, combining data of patients with high Oncotype scores (two of which are among those with relapse; FIG. 5B), they observed a discrete partition of patients with high risk of relapse (FIGS. 5A, B).

Thus, according to a first aspect of the present invention, there is provided a method of diagnosing breast cancer in a subject, comprising:

(a) isolating extracellular vesicles from serum and/or plasma of the subject to generate an isolated population of extracellular vesicles;

(b) lysing said isolated population of extracellular vesicles to generate a composition comprising components of said extracellular vesicles;

(c) measuring the amount of MEK1 in said composition; and

(d) diagnosing the subject with breast cancer when a level of said MEK1 in said composition is above a predetermined threshold. As used herein, the term “diagnosing” refers to determining presence or absence of the disease, classifying the disease, determining a severity of the disease, monitoring disease progression, forecasting an outcome of a pathology and/or prospects of recovery and/or screening of a subject for the cancer.

Ruling in the breast cancer refers to determining that the subject has breast cancer.

Ruling out the breast cancer refers to determining that the subject does not have breast cancer.

As used herein, the term “breast cancer” refers to any type of breast cancer at all stages of progression. The earliest stage breast cancers are called stage 0 (a pre-cancerous condition, either ductal carcinoma in situ or lobular carcinoma in situ), and then range from stage I through IV. In stage IV of breast cancer, also known as metastatic breast cancer, the cancer has spread beyond the breast and regional lymph nodes. The staging system most often used for breast cancer is the American Joint Committee on Cancer (AJCC) TNM system, which is based on the size of the tumor, the spread to the lymph nodes in the armpits, and whether the tumor has metastasized.

In one embodiment, the breast cancer is a metastatic breast cancer.

As used herein, the term “metastatic cancer” refers to cancer cells which break away from where they first formed and travel through the blood or lymph system to form new tumors (called metastatic tumor or metastasis) in other parts of the body.

According to a particular embodiment, the breast cancer is triple negative breast cancer.

The subject is typically a mammalian subject—e.g. a human female subject.

As used herein, the term “extracellular vesicle” or “EV” refers to a cell-derived vesicle comprising a membrane that encloses an internal space. Extracellular vesicles comprise all membrane-bound vesicles (e.g., exosomes or nanovesicle) that have a smaller diameter than the cell from which they are derived. In some aspects, extracellular vesicles range in diameter from nm to 1000 nm, and may comprise various macromolecular cargo either within the internal space (i.e., lumen), displayed on the external surface of the extracellular vesicle, and/or spanning the membrane. Components of extracellular vesicles include nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane).

The term “exosomes” as used herein refers to externally released vesicles originating from the endosomic compartment of cells. Exosomes typically have a particle size of about 20-200 nm (e.g. about 30-150 nm) and are released from many different cell types, including but not limited to, tumor cells, red blood cells, platelets, immune cells (e.g. antigen presenting cells, dendritic cells, macrophages, mast cells, T lymphocytes or B lymphocytes), kidney cells, hepatic cells, cardiac cells, lung cells, spleen cells, pancreatic cells, brain cells, skin cells, mesenchymal stem cells (e.g. human umbilical cord MSCs) and other cell types.

Typically, exosomes are formed by invagination and budding from the limiting membrane of late endosomes. They accumulate in cytosolic multivesicular bodies (MVBs) from where they are released by fusion with the plasma membrane. Alternatively, vesicles similar to exosomes (though somewhat larger, often called ‘microvesicles’) can be released directly from the plasma membrane. The process of vesicle shedding is particularly active in proliferating cells, such as cancer cells, where the release can occur continuously. Depending on the cellular origin, exosomes harbor biological material including e.g. nucleic acids (e.g. RNA or DNA), proteins, peptides, polypeptides, antigens, lipids, carbohydrates, and proteoglycans. For example, various cellular proteins can be found in exosomes including MHC molecules, tetraspanins, adhesion molecules and metalloproteinases.

The volume of the biological sample used for analyzing extracellular vesicles (e.g. exosomes) can be in the range of between 0.1-100 mL, such as less than about 100, 75, 50, 25, 15, 10, 9, 8, 7, 6, 5, 4, 3, 2, 1 or 0.1 mL.

The biological sample of some embodiments of the invention may comprise any number of extracellular vesicles (e.g. exosomes), e.g. 1, 5, 10, 15, 20, 25, 50, 100, 150, 200, 250, 500, 1000, 2000, 5000, 10,000, 50,000, 100,000, 500,000, lx106 or more exosomes.

As used herein, the term “exosome fraction” relates to a fraction of the biological sample comprising the exosomes.

In one embodiment, the exosome fraction is depleted of serum albumin compared to the non-exosome fraction.

According to one embodiment, the exosome fraction comprises exosomes and is free of intact cells.

According to one embodiment, exosomes are obtained from a freshly collected biological sample or from a biological sample that has been stored frozen or refrigerated.

Exosomes can be isolated from the biological sample by any method known in the art. Suitable methods are taught, for example, in U.S. Pat. Nos. 9,347,087 and 8,278,059, incorporated herein by reference.

For example, exosomes may be purified or concentrated from a biological sample using size exclusion chromatography, density gradient centrifugation, differential centrifugation, nanomembrane ultrafiltration, immunoabsorbent capture, affinity purification, microfluidic separation, or combinations thereof.

Size exclusion chromatography, such as gel permeation columns, centrifugation or density gradient centrifugation, and filtration methods can be used. For example, exosomes can be isolated by differential centrifugation, anion exchange and/or gel permeation chromatography (as described e.g. in U.S. Pat. Nos. 6,899,863 and 6,812,023), sucrose density gradients, organelle electrophoresis (as described e.g. in U.S. Pat. No. 7,198,923), magnetic activated cell sorting (MACS), or with a nanomembrane ultrafiltration concentrator. Thus, various combinations of isolation or concentration methods can be used as known to one of skill in the art.

Sub-populations of exosomes may also be isolated by using other properties of the exosomes such as the presence of surface markers. Surface markers which may be used for fraction of exosomes include but are not limited to tumor markers, cell type specific markers and MHC class II markers. MHC class II markers which have been associated with exosomes include HLA DP, DQ and DR haplotypes. Other surface markers associated with exosomes include, but are not limited to, CD9, CD81, CD63, CD82, CD37, CD53, or Rab-5b (Thery et al. Nat. Rev. Immunol. 2 (2002) 569-579; Valadi et al. Nat. Cell. Biol. 9 (2007) 654-659).

Determining the amount of exosomes in a sample can be carried out using any method known in the art, e.g. by ELISA, using commercially available kits such as, for example, the ExoQuick kit (System Biosciences, Mountain View, Calif.), magnetic activated cell sorting (MACS) or by FACS using an antigen or antigens which bind general exosome markers, such as but not limited to, CD63, CD9, CD81, CD82, CD37, CD53, or Rab-5b.

In one embodiment, the exosomes are purified to minimize the amount of contamination with plasma proteins (e.g. serum albumin). This may be carried out using at least one of the above described methods—e.g. filtration and size exclusion chromatography (SEC). According to a particular embodiment the method does not include an ultracentrifugation step. Size exclusion chromatography may be carried out in a number of fractions and the particular fraction which is enriched in exosomes but not in plasma proteins may be selected. In one embodiment, the purifying is carried out by depleting the composition of serum albumin. The abundance of particles of a particular size in each fraction can be analyzed by light scattering (e.g. NanoSight). The abundance of albumin in the isolated fractions compared to total plasma can be assessed by methods known in the art e.g. Coomassie Blue staining of similar lysate volumes.

According to one embodiment, once an isolated exosome sample (i.e. exosome fraction) has been prepared it can be stored, such as in a sample bank and retrieved for analysis as necessary, alternatively, the exosome fraction can be analyzed without storing the sample.

According to another embodiment, the contents of the exosomes are extracted for study and characterization. Biological material which may be extracted from exosomes includes, for example, proteins, peptides, polypeptides, nucleic acids (e.g. RNA or DNA) and lipids. For example the mirVana.TM. PARIS Kit (AM1556, Life Technologies) or the ME™ Kit for Exosome Isolation may be used to recover native protein and RNA species, including small RNAs such as miRNA, snRNA, and snoRNA, from exosomes.

Detection of an activity or expression of any of the disclosed protein markers (i.e. disease determinants) in an exosome fraction can be carried out using any method known in the art, e.g. on the polypeptide level or on the RNA level.

Following is a non-limiting list of examples of methods of determining the activity or expression of one of the disclosed proteins on the polypeptide level. It will be appreciated that when the marker is analyzed on the protein level, a protein extract is prepared from the isolated extracellular vesicles. To prepare a protein extract, the vesicles are lysed in an appropriate buffer. The buffer may comprise protease inhibitors to protect the proteins therein from degradation.

Typically, analyzing the level of proteins involves the use of antibodies that specifically bind to the particular protein. The antibody may be monoclonal, polyclonal, chimeric, or a fragment of the foregoing, and the step of detecting the protein determinant may be carried out with any suitable immunoassay. Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵ I¹³¹ I) enzyme labels (e.g., horseradish peroxidase, alkaline phosphatase), and fluorescent labels (e.g., fluorescein, Alexa, green fluorescent protein, rhodamine) in accordance with known techniques.

Enzyme linked immunosorbent assay (ELISA): This method involves a reaction between an enzyme and a substrate. A biological sample which comprises a component of the necroptosis activation pathway (e.g. exosome fraction disrupted using detergent) is put in a microwell dish. A specific antibody (e.g. capable of targeting a component of the necroptosis activation pathway) coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate (e.g. an antibody capable of targeting a component of the necroptosis activation pathway), which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody capable of targeting a component of the necroptosis activation pathway, and radiolabeled antibody binding protein (e.g., protein A labeled with I.sup.125) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in exosomes by substrate specific antibodies i.e., antibodies capable of targeting one of the disclosed protein markers. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed exosomes by substrate specific antibodies, i.e., antibodies capable of targeting one of the disclosed protein markers. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the exosomes containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the exosomes. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

Mass spectrometry based techniques, forward and reverse phase protein arrays are also contemplated.

Following is a non-limiting list of examples of methods of determining the expression of the disclosed proteins on the polynucleotide level.

It will be appreciated that when the marker is analyzed on the polynucleotide level, an RNA extract is prepared from the isolated extracellular vesicles. To prepare an RNA extract, the vesicles are lysed in an appropriate buffer. The buffer typically includes phenol and/or guanidine isothiocyanate. The buffer may comprise RNAse inhibitors to protect the RNA therein from degradation. Typically cDNA is prepared from the RNA sample using a reverse transcriptase enzyme and primers such as, oligo dT, random hexamers or gene specific primers.

The presence and/or level of one of the disclosed proteins can be determined using an isolated polynucleotide (e.g., a polynucleotide probe, an oligonucleotide probe/primer) capable of hybridizing to a nucleic acid sequence of one of the determinants described herein. Such a polynucleotide can be at any size, such as a short polynucleotide (e.g., of 15-200 bases), and intermediate polynucleotide (e.g., 200-2000 bases) or a long polynucleotide larger of 2000 bases.

The isolated polynucleotide probe used by the present invention can be any directly or indirectly labeled RNA molecule (e.g., RNA oligonucleotide, an in vitro transcribed RNA molecule), DNA molecule (e.g., oligonucleotide, cDNA molecule, genomic molecule) and/or an analogue thereof [e.g., peptide nucleic acid (PNA)] which is specific to the RNA transcript of the present invention.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The above-described polynucleotides can be employed in a variety of transcript detection methods. Following is a non-limiting list of RNA-based hybridization methods which can be used to detect the protein markers of the present invention.

Northern Blot analysis—This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA, RNA or oligonucleotide (composed of deoxyribo or ribonucleotides) probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

Reverse-transcribed PCR (RT-PCR) analysis—This method is performed using specific primers. It will be appreciated that a semi-quantitative RT-PCR reaction can be also employed by adjusting the number of PCR cycles and comparing the amplification product to known controls. Alternatively, quantitative RT-PCR can be performed using, for example, the Light Cycler.TM. (Roche).

RNA in situ hybridization stain—In this method DNA, RNA or oligonucleotide (composed of deoxyribo or ribonucleotides) probes are attached to the RNA molecules present in the exosomes. Generally, the exosomes are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of exosomes. Following hybridization, any unbound probe is washed off and the slide is subjected to either a photographic emulsion which reveals signals generated using radio-labeled probes or to a colorimetric reaction which reveals signals generated using enzyme-linked labeled probes.

Oligonucleotide microarray analysis—This method can be performed by attaching oligonucleotide probes which are capable of specifically hybridizing with the transcript of one of the disclosed proteins to a solid surface (e.g., a glass wafer). Each oligonucleotide probe is of approximately 20-25 nucleic acids in length. To detect the expression pattern of the transcript of the necroptosis activation pathway of the present invention in a specific sample (e.g., exosomes), RNA is extracted from the exosomes using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94.degree. C. Following hybridization, the microarray is washed and the hybridization signal is scanned using a confocal laser fluorescence scanner which measures fluorescence intensity emitted by the labeled cRNA bound to the probe arrays.

Affymetrix microarray (Affymetrix^{RTM}, Santa Clara, Calif.)—in this method each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

As mentioned, in one aspect, a subject is diagnosed as having breast cancer based on the level of the determinant “MEK1” in the extracellular vesicles.

The term “MEK1” refers to mitogen/extracellular signal-regulated kinase-1 having the UniProt ID: Q02750; Entrez-Gene Id: 5604). In one embodiment, the term “MEK1” refers to a splice variant or cancer-associated mutation thereof.

According to a particular embodiment, the MEK1 is analyzed on the protein level. In order to measure MEK1 on the protein level, an antibody may be used which specifically binds to MEK1 (e.g. the antibody binds specifically to MEK1 and not to MEK2). An exemplary antibody which can be used to measure the amount of MEK1 in the sample is available at Cell Signaling Technology—Catalogue No. #9124 or Santa Cruz Technologies (sc-6250).

According to another embodiment, the MEK1 is analyzed on the RNA level. In order to measure MEK1 on the RNA level, primer pairs can be used which bind specifically to the cDNA sample prepared from the isolated population of extracellular vesicles. An exemplary primer pair is provided herein below:

Forward sequence:
(SEQ ID NO: 1)
GGTGTTCAAGGTCTCCCACAAG
Reverse sequence:
(SEQ ID NO: 2)
CCACGATGTACGGAGAGTTGCA.

A subject may be diagnosed as having breast cancer when the level of MEK1 is above a predetermined level. Typically, the predetermined level is at least 1.5 times higher, at least two times higher, at least five times higher or even at least 10 times higher than the level of MEK1 found in the extracellular vesicles of a control healthy subject.

The present inventors contemplate analyzing the levels of additional markers in the extracellular vesicles of the test subject to increase the accuracy of the diagnosis.

Thus, for example the present inventors contemplate analyzing the level of fibronectin and/or FAK in the extracellular vesicles.

The term “fibronectin” (also referred to as fibronectin 1) has the UniProt ID: P02751; Entrez-Gene Id: 2335). In one embodiment, the term fibronectin refers to a splice variant or cancer-associated mutation thereof.

According to a particular embodiment, fibronectin is analyzed on the protein level. In order to measure fibronectin on the protein level, an antibody may be used which specifically binds to fibronectin. An exemplary antibody which can be used to measure the amount of fibronectin in the sample is available at LifeSpan BioSciences—Catalogue No. LS-B7080 or

Invitrogen (AFLGC-FN1).

According to another embodiment, the fibronectin is analyzed on the RNA level. In order to measure fibronectin on the RNA level, primer pairs can be used which bind specifically to the cDNA sample prepared from the isolated population of extracellular vesicles. An exemplary primer pair is provided herein below:

Forward sequence:
(SEQ ID NO: 3)
5′-CCA TCG CAA ACC GCT GCC AT-3′
Reverse sequence:
(SEQ ID NO: 4)
5′-AAC ACT TCT CAG CTA TGG GCT T-3′.

A subject may be diagnosed as having breast cancer when the level of MEK1 is above a predetermined level and when the level of fibronectin is above a predetermined level. Typically, the predetermined level for MEK1 is at least 1.5 times higher, at least two times higher, at least five times higher or even at least 10 times higher than the level of MEK1 found in the extracellular vesicles of a control healthy subject. Typically, the predetermined level for fibronectin is at least 1.5 times higher, at least two times higher, at least five times higher or even at least 10 times higher than the level of fibronectin found in the extracellular vesicles of a control healthy subject.

The term “FAK” refers to focal adhesion kinase having the UniProt ID: Q05397; Entrez-Gene Id: 5747). In one embodiment, the term “REK” refers to a splice variant or cancer-associated mutation thereof.

According to a particular embodiment, FAK is analyzed on the protein level. In order to measure fibronectin on the protein level, an antibody may be used which specifically binds to FAK. An exemplary antibody which can be used to measure the amount of FAK in the sample is available at Santa Cruz—Catalogue No. SC-932 or LifeSpan BioSciences (LS-A3392).

According to another embodiment, the FAK is analyzed on the RNA level. In order to measure FAK on the RNA level, primer pairs can be used which bind specifically to the cDNA sample prepared from the isolated population of extracellular vesicles. An exemplary primer pair is provided herein below:

Exemplary primers for measuring FAK include those detailed in Corsi et al.,BMC Genomics. 2006; 7: 198.

A subject may be diagnosed as having breast cancer when the level of MEK1 is above a predetermined level and when the level of FAK is above a predetermined level. Typically, the predetermined level for MEK1 is at least 1.5 times higher, at least two times higher, at least five times higher or even at least 10 times higher than the level of MEK1 found in the extracellular vesicles of a control healthy subject. Typically, the predetermined level for FAK is at least 1.5 times higher, at least two times higher, at least five times higher or even at least 10 times higher than the level of fibronectin found in the extracellular vesicles of a control healthy subject.

In another embodiment, each of MEK1, fibronectin and FAK are measured and used to make the diagnosis.

Additional determinants that can be used to rule in (i.e. diagnose) whether a subject has breast cancer include β-Actin (Uniprot P60709), C-Raf (Uniprot P04049), N-Cadherin (Uniprot 19022) and P90RSK_pT573 (the phosphorylated form of MAPKAPK2 —Entrez ID 9261. Measurement of these determinants may be carried out on the protein level of the polynucleotide level, as further detailed herein above.

A subject may be diagnosed as having breast cancer when the level of β-actin, C-Raf, P90RSK_pT573 and/or N-cadherin is below a predetermined level. Typically, the predetermined level for these proteins is at least 1.5 times lower, at least two times lower, at least five times lower or even at least 10 times lower than their level found in the extracellular vesicles of a control healthy subject.

In one embodiment, at least one, two, three or all of the proteins in the group which includes β-Actin (Uniprot P60709), C-Raf (Uniprot P04049), N-Cadherin (Uniprot 19022) and P90RSK_pT573 is used to diagnose breast cancer (together with MEK1, fibronectin and FAK).

According to another aspect of the present invention there is provided a method of staging breast cancer in a subject in need thereof, comprising:

(a) isolating extracellular vesicles (e.g. exosomes) from serum and/or plasma of the subject to generate an isolated population of extracellular vesicles;

(b) lysing said extracellular vesicles to generate a composition comprising isolated components of said extracellular vesicles;

(c) measuring the amount of at least one protein selected from the group consisting of P-Cadherin, TAZ, cleaved caspase-7, EGFR, E2F1, Aurora-B, IGFRβ, NF-κB-p65 in said composition; and

(d) staging the breast cancer according to the amount.

Details of each of the above disclosed proteins are provided herein below.

P-Cadherin—Uniprot No. P22223 (exemplary antibody is commercially available from Abcam—Catalogue No. ab242060),

TAZ—Uniprot No. Q16635 (exemplary antibody is commercially available from Abcam—Catalogue No. ab176396),

cleaved caspase-7 —Uniprot No. P55210 (exemplary antibody is commercially available from Abcam—Catalogue No. ab69876),

EGFR—Uniprot No. P00533 (exemplary antibody is commercially available from Abcam—Catalogue No. ab52894),

E2F1 —Uniprot No. Q01094 (exemplary antibody is commercially available from Abcam—Catalogue No. ab4070),

Aurora-B—Uniprot No. Q96GD4 (exemplary antibody is commercially available from Abcam—Catalogue No. ab45145),

IGFRβ—Uniprot No. P08069 (exemplary antibody is commercially available from Abcam—Catalogue No. ab182408),

NF-κB-p65 —Uniprot No. Q04206 (exemplary antibody is commercially available from Abcam—Catalogue No. ab32518).

Stages of Breast Cancer:

Measuring of these determinants can be carried out on the protein level or the polynucleotide level as further described herein above. Examples of extracellular vesicles are also described herein above.

The term “staging” refers to identifying the stage to which the disease has progressed.

According to a particular embodiment, the staging is according to that set by the American Joint Committee on Cancer (AJCC).

The staging takes into account at least three clinical characteristics T, N, and M:

the size of the cancer tumor and whether or not it has grown into nearby tissue (T)

whether cancer is in the lymph nodes (N)

whether the cancer has spread to other parts of the body beyond the breast (M).According to the AJCC staging system, stage 0 is used to describe non-invasive breast cancers, such as DCIS (ductal carcinoma in situ). In stage 0, there is no evidence of cancer cells or non-cancerous abnormal cells breaking out of the part of the breast in which they started, or getting through to or invading neighboring normal tissue.

Stage I describes invasive breast cancer (cancer cells are breaking through to or invading normal surrounding breast tissue) Stage I is divided into subcategories known as IA and IB. In general, stage IA describes invasive breast cancer in which the tumor measures up to 2 centimeters (cm) and the cancer has not spread outside the breast; no lymph nodes are involved.

In general, stage IB describes invasive breast cancer in which there is no tumor in the breast; instead, small groups of cancer cells—larger than 0.2 millimeter (mm) but not larger than 2 mm—are found in the lymph nodes or there is a tumor in the breast that is no larger than 2 cm, and there are small groups of cancer cells—larger than 0.2 mm but not larger than 2 mm —in the lymph nodes

Stage II is divided into subcategories known as IIA and IIB.

In general, stage IIA describes invasive breast cancer in which no tumor can be found in the breast, but cancer (larger than 2 millimeters [mm]) is found in 1 to 3 axillary lymph nodes (the lymph nodes under the arm) or in the lymph nodes near the breast bone (found during a sentinel node biopsy) or the tumor measures 2 centimeters (cm) or smaller and has spread to the axillary lymph nodes or the tumor is larger than 2 cm but not larger than 5 cm and has not spread to the axillary lymph nodes.

In general, stage IIB describes invasive breast cancer in which the tumor is larger than 2 cm but no larger than 5 centimeters; small groups of breast cancer cells—larger than 0.2 mm but not larger than 2 mm—are found in the lymph nodes or the tumor is larger than 2 cm but no larger than 5 cm; cancer has spread to 1 to 3 axillary lymph nodes or to lymph nodes near the breastbone (found during a sentinel node biopsy) or the tumor is larger than 5 cm but has not spread to the axillary lymph nodes.

Stage III is divided into subcategories known as IIIA, IIIB, and IIIC. In general, stage IIIA describes invasive breast cancer in which either: no tumor is found in the breast or the tumor may be any size; cancer is found in 4 to 9 axillary lymph nodes or in the lymph nodes near the breastbone (found during imaging tests or a physical exam) or the tumor is larger than 5 centimeters (cm); small groups of breast cancer cells (larger than 0.2 millimeter [mm] but not larger than 2 mm) are found in the lymph nodes or the tumor is larger than 5 cm; cancer has spread to 1 to 3 axillary lymph nodes or to the lymph nodes near the breastbone (found during a sentinel lymph node biopsy).

In general, stage IIIB describes invasive breast cancer in which the tumor may be any size and has spread to the chest wall and/or skin of the breast and caused swelling or an ulcer and may have spread to up to 9 axillary lymph nodes or may have spread to lymph nodes near the breastbone.

Inflammatory breast cancer is considered at least stage IIIB. Typical features of inflammatory breast cancer include: reddening of a large portion of the breast skin, the breast feels warm and may be swollen or cancer cells have spread to the lymph nodes and may be found in the skin.

In general, stage IIIC describes invasive breast cancer in which there may be no sign of cancer in the breast or, if there is a tumor, it may be any size and may have spread to the chest wall and/or the skin of the breast and the cancer has spread to 10 or more axillary lymph nodes or the cancer has spread to lymph nodes above or below the collarbone or the cancer has spread to axillary lymph nodes or to lymph nodes near the breastbone.

Stage IV describes invasive breast cancer that has spread beyond the breast and nearby lymph nodes to other organs of the body, such as the lungs, distant lymph nodes, skin, bones, liver, or brain.

In one embodiment, at least two of the above proteins are measured and used to stage the breast cancer. In another embodiment, at least three of the above proteins are measured and used to stage the breast cancer. In still another embodiment, at least four of the above proteins are measured and used to stage the breast cancer. In another embodiment, at least five of the above proteins are measured and used to stage the breast cancer. In another embodiment, at least six of the above proteins are measured and used to stage the breast cancer. In another embodiment, seven of the above proteins are measured and used to stage the breast cancer. In another embodiment, all of the above proteins are measured and used to stage the breast cancer.

The level of P-Cadherin, TAZ, cleaved caspase-7 typically decreases according to the stage of the breast cancer—thus a low level of one of these proteins is indicative of a late stage breast cancer (i.e. later than stage I) and/or a high level of one of these proteins is indicative of an early stage breast cancer (e.g. stage I).

The level of EGFR, E2F1, Aurora-B typically increases according to the stage of the breast cancer—thus a low level of one of these proteins is indicative of an early stage breast cancer (e.g. stage I) and/or a high level of one of these proteins is indicative of a later stage breast cancer (e.g. later than stage I).

In one embodiment, both IGFRβ and NF-κB-p65 are measured. These proteins typically decrease according to the stage of the breast cancer and are particularly useful at distinguishing between stage I and stage IIA cancer. Thus a low level of each of these proteins is indicative of a late stage breast cancer (i.e. later than stage I) and/or a high level of one of these proteins is indicative of an early stage breast cancer (e.g. stage I).

As well as measuring the proteins described herein above, the present inventors further contemplate analyzing the size distribution of extracellular vesicles derived from the serum and/or plasma of the subject in order to stage the cancer. In general, the lower the size distribution of the extracellular vesicles, the later the stage of the breast cancer.

Methods of analyzing the size distribution of the extracellular vesicles are known in the art and include for example dynamic light scattering techniques.

According to still another aspect of the present invention there is provided a method of determining the risk of breast cancer relapse in a subject, comprising:
(a) isolating extracellular vesicles from serum and/or plasma of the subject to generate an isolated population of extracellular vesicles;
(b) lysing said extracellular vesicles to generate a composition comprising components of said extracellular vesicles;
(c) measuring an expression level of at least one of the proteins selected from the group consisting of MIF, COG3, Cox-IV, cyclophilin-F, EMA, HSP70, MMP2 and VEGFR-2 in said composition, wherein the expression level of said at least one of said proteins correlates with the risk of breast cancer relapse.

The subjects of this aspect of the present invention typically have already had breast cancer and have either partially or fully recovered from said cancer.

For this aspect, the amount of at least one of the proteins listed below is measured and used to determine whether the subject is likely to relapse or not.

MIF (UniProt P14174; Entrez Gene 4282),

COG3 (UniProt Q96JB2; Entrez Gene 83548),

Cox-IV (Unipot P13073, Entrez gene 1327)

cyclophilin-F (Uniprot P30405, Entrez gene 10105)

EMA (Ubiorot P15941, Enzrez gene 4582),

HSP70 (UniProt PODMV8; Entrez Gene 3303),

MMP2 (UniProt P08253; Entrez Gene 4313)

VEGFR-2 (UniProt P35968; Entrez Gene 3791).

For this aspect, when the level of MIF, COG3, Cox-IV, cyclophilin-F, EMA or HSP70 is above a predetermined threshold, it is indicative of a high risk for breast cancer relapse. When the level of MIF, COG3, Cox-IV, cyclophilin-F, EMA or HSP70 is below a predetermined threshold, it is indicative of a low risk for breast cancer relapse. When the level of MMP2 and VEGFR-2 is below a predetermined threshold, it is indicative of a high risk of breast cancer relapse. When the level of MMP2 and VEGFR-2 is above a predetermined threshold, it is indicative of a high risk of breast cancer relapse.

For any of the aspects described herein above, once the diagnosis has been made, additional tests may be undertaken to corroborate the result. The additional tests may include for example imaging (mammogram, ultrasound, MRI) or taking a biopsy.

Once a diagnosis has been made, the clinician can treat accordingly. Depending on the stage of the cancer, the clinician may decide to treat more or less aggressively. Depending on the results of the testing, the clinician may decide to treat surgically (e.g. performing a lumpectomy, partial mastectomy or radical mastectomy).

Exemplary chemotherapeutic agents that can be used to treat breast cancer include: Docetaxel (Taxotere), Paclitaxel (Taxol), Doxorubicin, Epirubicin (Ellence), Pegylated liposomal doxorubicin (Doxil), Capecitabine (Xeloda), Carboplatin, Cisplatin, Cyclophosphamide, Eribulin (Halaven), Fluorouracil (5-FU), Gemcitabine (Gemzar), Ixabepilone (Ixempra) and Methotrexate.

Hormonal therapies for the treatment of breast cancer include tamoxifen and Aromatase inhibitors.

Construction of Clinical Algorithms:

Often, for binary disease state classification approaches using a continuous diagnostic test measurement, the sensitivity and specificity is summarized by a Receiver Operating Characteristics (ROC) curve according to Pepe et al, “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4th edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), Matheus correlation coefficient (MCC), or as a likelihood, odds ratio, Receiver Operating Charachteristic (ROC) curve, Area Under the Curve (AUC) among other measures.

In order to uncover an algorithm that can be used to accurately diagnose a subject with breast cancer (including staging and prediction of relapse), in accordance with embodiments of the present invention, a machine learning procedure can be executed.

As used herein the term “machine learning” refers to a procedure embodied as a computer program configured to induce patterns, regularities, or rules from previously collected data to develop an appropriate response to future data, or describe the data in some meaningful way.

Use of machine learning is particularly, but not exclusively, advantageous when the input dataset includes multidimensional entries.

In machine learning, information can be acquired via supervised learning or unsupervised learning. In some embodiments of the invention the machine learning procedure comprises, or is, a supervised learning procedure. In supervised learning, global or local goal functions are used to optimize the structure of the learning system. In other words, in supervised learning there is a desired response, which is used by the system to guide the learning.

In some embodiments of the invention the machine learning procedure comprises, or is, an unsupervised learning procedure. In unsupervised learning there are typically no goal functions. In particular, the learning system is not provided with a set of rules. One form of unsupervised learning according to some embodiments of the present invention is unsupervised clustering in which the data objects are not class labeled, a priori.

Representative examples of “machine learning” procedures suitable for the present embodiments, including, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors analysis, ensemble learning algorithms, probabilistic models, graphical models, logistic regression methods (including multinomial logistic regression methods), gradient ascent methods, singular value decomposition methods and principle component analysis. Among neural network models, the self-organizing map and adaptive resonance theory are commonly used unsupervised learning algorithms. The adaptive resonance theory model allows the number of clusters to vary with problem size and lets the user control the degree of similarity between members of the same clusters by means of a user-defined constant called the vigilance parameter.

Following is an overview of some machine learning procedures suitable for the present embodiments.

Association rule algorithm is a technique for extracting meaningful association patterns among features.

The term “association”, in the context of machine learning, refers to any interrelation among features, not just ones that predict a particular class or numeric value. Association includes, but it is not limited to, finding association rules, finding patterns, performing feature evaluation, performing feature subset selection, developing predictive models, and understanding interactions between features.

The term “association rules” refers to elements that co-occur frequently within the datasets. It includes, but is not limited to association patterns, discriminative patterns, frequent patterns, closed patterns, and colossal patterns.

A usual primary step of association rule algorithm is to find a set of items or features that are most frequent among all the observations. Once the list is obtained, rules can be extracted from them.

The aforementioned self-organizing map is an unsupervised learning technique often used for visualization and analysis of high-dimensional data. Typical applications are focused on the visualization of the central dependencies within the data on the map. The map generated by the algorithm can be used to speed up the identification of association rules by other algorithms. The algorithm typically includes a grid of processing units, referred to as “neurons”. Each neuron is associated with a feature vector referred to as observation. The map attempts to represent all the available observations with optimal accuracy using a restricted set of models. At the same time the models become ordered on the grid so that similar models are close to each other and dissimilar models far from each other. This procedure enables the identification as well as the visualization of dependencies or associations between the features in the data.

Feature evaluation algorithms are directed to the ranking of features or to the ranking followed by the selection of features based on their impact.

The term “feature” in the context of machine learning refers to one or more raw input variables, to one or more processed variables, or to one or more mathematical combinations of other variables, including raw variables and processed variables. Features may be continuous or discrete.

Information gain is one of the machine learning methods suitable for feature evaluation. The definition of information gain requires the definition of entropy, which is a measure of impurity in a collection of training instances. The reduction in entropy of the target feature that occurs by knowing the values of a certain feature is called information gain. Information gain may be used as a parameter to determine the effectiveness of a feature in explaining the cancer diagnosis. Symmetrical uncertainty is an algorithm that can be used by a feature selection algorithm, according to some embodiments of the present invention. Symmetrical uncertainty compensates for information gain's bias towards features with more values by normalizing features to a [0,1] range.

Subset selection algorithms rely on a combination of an evaluation algorithm and a search algorithm. Similarly to feature evaluation algorithms, subset selection algorithms rank subsets of features. Unlike feature evaluation algorithms, however, a subset selection algorithm suitable for the present embodiments aims at selecting the subset of features with the highest impact on the cancer diagnosis, while accounting for the degree of redundancy between the features included in the subset. The benefits from feature subset selection include facilitating data visualization and understanding, reducing measurement and storage requirements, reducing training and utilization times, and eliminating distracting features to improve classification.

Two basic approaches to subset selection algorithms are the process of adding features to a working subset (forward selection) and deleting from the current subset of features (backward elimination). In machine learning, forward selection is done differently than the statistical procedure with the same name. The feature to be added to the current subset in machine learning is found by evaluating the performance of the current subset augmented by one new feature using cross-validation. In forward selection, subsets are built up by adding each remaining feature in turn to the current subset while evaluating the expected performance of each new subset using cross-validation. The feature that leads to the best performance when added to the current subset is retained and the process continues. The search ends when none of the remaining available features improves the predictive ability of the current subset. This process finds a local optimum set of features.

Backward elimination is implemented in a similar fashion. With backward elimination, the search ends when further reduction in the feature set does not improve the predictive ability of the subset. The present embodiments contemplate search algorithms that search forward, backward or in both directions. Representative examples of search algorithms suitable for the present embodiments include, without limitation, exhaustive search, greedy hill-climbing, random perturbations of subsets, wrapper algorithms, probabilistic race search, schemata search, rank race search, and Bayesian classifier.

A decision tree is a decision support algorithm that forms a logical pathway of steps involved in considering the input to make a decision.

The term “decision tree” refers to any type of tree-based learning algorithms, including, but not limited to, model trees, classification trees, and regression trees.

A decision tree can be used to classify the datasets or their relation hierarchically. The decision tree has tree structure that includes branch nodes and leaf nodes. Each branch node specifies an attribute (splitting attribute) and a test (splitting test) to be carried out on the value of the splitting attribute, and branches out to other nodes for all possible outcomes of the splitting test. The branch node that is the root of the decision tree is called the root node. Each leaf node can represent a classification (e.g., whether a particular portion of the group dataset matches a particular portion of the subject-specific dataset) or a value. The leaf nodes can also contain additional information about the represented classification such as a confidence score that measures a confidence in the represented classification (i.e., the likelihood of the classification being accurate). For example, the confidence score can be a continuous value ranging from 0 to 1, which a score of 0 indicating a very low confidence (e.g., the indication value of the represented classification is very low) and a score of 1 indicating a very high confidence (e.g., the represented classification is almost certainly accurate).

Support vector machines are algorithms that are based on statistical learning theory. A support vector machine (SVM) according to some embodiments of the present invention can be used for classification purposes and/or for numeric prediction. A support vector machine for classification is referred to herein as “support vector classifier,” support vector machine for numeric prediction is referred to herein as “support vector regression”.

An SVM is typically characterized by a kernel function, the selection of which determines whether the resulting SVM provides classification, regression or other functions. Through application of the kernel function, the SVM maps input vectors into high dimensional feature space, in which a decision hyper-surface (also known as a separator) can be constructed to provide classification, regression or other decision functions. In the simplest case, the surface is a hyper-plane (also known as linear separator), but more complex separators are also contemplated and can be applied using kernel functions. The data points that define the hyper-surface are referred to as support vectors.

The support vector classifier selects a separator where the distance of the separator from the closest data points is as large as possible, thereby separating feature vector points associated with objects in a given class from feature vector points associated with objects outside the class. For support vector regression, a high-dimensional tube with a radius of acceptable error is constructed which minimizes the error of the data set while also maximizing the flatness of the associated curve or function. In other words, the tube is an envelope around the fit curve, defined by a collection of data points nearest the curve or surface.

An advantage of a support vector machine is that once the support vectors have been identified, the remaining observations can be removed from the calculations, thus greatly reducing the computational complexity of the problem. An SVM typically operates in two phases: a training phase and a testing phase. During the training phase, a set of support vectors is generated for use in executing the decision rule. During the testing phase, decisions are made using the decision rule. A support vector algorithm is a method for training an SVM. By execution of the algorithm, a training set of parameters is generated, including the support vectors that characterize the SVM. A representative example of a support vector algorithm suitable for the present embodiments includes, without limitation, sequential minimal optimization.

Regression techniques which may be used in accordance with the present invention include, but are not limited to linear Regression, Multiple Regression, logistic regression, probit regression, ordinal logistic regression ordinal Probit-Regression, Poisson Regression, negative binomial Regression, multinomial logistic Regression (MLR) and truncated regression.

A logistic regression or logit regression is a type of regression analysis used for predicting the outcome of a categorical dependent variable (a dependent variable that can take on a limited number of values, whose magnitudes are not meaningful but whose ordering of magnitudes may or may not be meaningful) based on one or more predictor variables. Logistic regressions also include a multinomial variant. The multinomial logistic regression model, is a regression model which generalizes logistic regression by allowing more than two discrete outcomes. That is, it is a model that is used to predict the probabilities of the different possible outcomes of a categorically distributed dependent variable, given a set of independent variables (which may be real-valued, binary-valued, categorical-valued, etc.).

The advantage of logistic regression is that it assigns an interpretable measure of prediction confidence—a probability. For example, patients predicted of having a breast cancer with a probability of 75% and 99%, would both be assigned as being positive when using an SVM interpretation function but the fact that the latter has a higher probability would be masked. Assigning the likelihood level of confidence adds valuable clinical information that may affect clinical judgment.

The Least Absolute Shrinkage and Selection Operator (LASSO) algorithm is a shrinkage and/or selection algorithm for linear regression. The LASSO algorithm may minimizes the usual sum of squared errors, with a regularization, that can be an L1 norm regularization (a bound on the sum of the absolute values of the coefficients), an L2 norm regularization (a bound on the sum of squares of the coefficients), and the like. The LASSO algorithm may be associated with soft-thresholding of wavelet coefficients, forward stagewise regression, and boosting methods. The LASSO algorithm is described in the paper: Tibshirani, R, Regression Shrinkage and Selection via the Lasso, J. Royal. Statist. Soc B., Vol. 58, No. 1, 1996, pages 267-288, the disclosure of which is incorporated herein by reference.

A Bayesian network is a model that represents variables and conditional interdependencies between variables. In a Bayesian network variables are represented as nodes, and nodes may be connected to one another by one or more links. A link indicates a relationship between two nodes. Nodes typically have corresponding conditional probability tables that are used to determine the probability of a state of a node given the state of other nodes to which the node is connected. In some embodiments, a Bayes optimal classifier algorithm is employed to apply the maximum a posteriori hypothesis to a new record in order to predict the probability of its classification, as well as to calculate the probabilities from each of the other hypotheses obtained from a training set and to use these probabilities as weighting factors for future predictions of the type of cancer (e.g. stage, prediction of relapse). An algorithm suitable for a search for the best Bayesian network, includes, without limitation, global score metric-based algorithm. In an alternative approach to building the network, Markov blanket can be employed. The Markov blanket isolates a node from being affected by any node outside its boundary, which is composed of the node's parents, its children, and the parents of its children.

Instance-based algorithms generate a new model for each instance, instead of basing predictions on trees or networks generated (once) from a training set.

The term “instance”, in the context of machine learning, refers to an example from a dataset.

Instance-based algorithms typically store the entire dataset in memory and build a model from a set of records similar to those being tested. This similarity can be evaluated, for example, through nearest-neighbor or locally weighted methods, e.g., using Euclidian distances. Once a set of records is selected, the final model may be built using several different algorithms, such as the naive Bayes.

A machine-readable storage medium can comprise a data storage material encoded with machine readable data or data arrays which, when using a machine programmed with instructions for using said data, is capable of use for a variety of purposes. Measurements of effective amounts of the biomarkers of the invention and/or the resulting evaluation of risk from those biomarkers can implemented in computer programs executing on programmable computers, comprising, inter alia, a processor, a data storage system (including volatile and non-volatile memory and/or storage elements), at least one input device, and at least one output device.

Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein.

The health-related data management system of the invention may also be considered to be implemented as a computer-readable storage medium, configured with a computer program, where the storage medium so configured causes a computer to operate in a specific and predefined manner to perform various functions described herein.

The recorded output may include the assay results, findings, diagnoses, predictions and/or treatment recommendations. These may be communicated to technicians, physicians and/or patients, for example. In certain embodiments, computers will be used to communicate such information to interested parties, such as, patients and/or the attending physicians. Based on the output, the therapy administered to a subject can be modified.

In one embodiment, the output is presented graphically. In another embodiment, the output is presented numerically (e.g. as a probability). In another embodiment, the output is generated using a color index (for example in a bar display) where one color indicates bacterial infection and another color non-bacterial infection.

In some embodiments, the output is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, such as by providing a written report, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system. In certain embodiments of the methods of the disclosure, all or some of the method steps, including the assaying of samples, diagnosing of diseases, and communicating of assay results or diagnoses, may be carried out in diverse (e.g., foreign) jurisdictions.

Kits

Some aspects of the invention also include determinant detection reagents, (e.g. antibodies) packaged together in the form of a kit. The kit may contain in separate containers an antibody (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR, CD-ROM, etc.) for carrying out the assay may be included in the kit. The assay may for example be in the form of a sandwich ELISA as known in the art.

For example, determinant detection reagents can be immobilized on a solid matrix such as a porous strip to form at least one determinant detection site. The measurement or detection region of the porous strip may include a plurality of sites. A test strip may also contain sites for negative and/or positive controls. Alternatively, control sites can be located on a separate strip from the test strip. Optionally, the different detection sites may contain different amounts of immobilized detection reagents, e.g., a higher amount in the first detection site and lesser amounts in subsequent sites. Upon the addition of test sample, the number of sites displaying a detectable signal provides a quantitative indication of the amount of determinant present in the sample. The detection sites may be configured in any suitably detectable shape and are typically in the shape of a bar or dot spanning the width of a test strip.

Suitable sources for antibodies for the detection of determinants include commercially available sources such as, for example, Abazyme, Abnova, AssayPro, Affinity Biologicals, AntibodyShop, Aviva bioscience, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, against any of the polypeptide determinant described herein.

Polyclonal antibodies for measuring determinants include without limitation antibodies that were produced from sera by active immunization of one or more of the following: Rabbit, Goat, Sheep, Chicken, Duck, Guinea Pig, Mouse, Donkey, Camel, Rat and Horse.

Examples of detection agents that can be included in the kits, include without limitation: scFv, dsFv, Fab, sVH, F(ab′)2, Cyclic peptides, Haptamers, A single-domain antibody, Fab fragments, Single-chain variable fragments, Affibody molecules, Affilins, Nanofitins, Anticalins, Avimers, DARPins, Kunitz domains, Fynomers and Monobody.

In a particular embodiment, the kit includes detection agents (e.g. antibodies) that specifically detect no more than two, three, four, five, six, seven, eight, nine, ten, 11, 12, 13, 14, 15, 16, 17, 18, 19 or 20 determinants. Thus, for example, the kit may comprise 3, 5, 7, 10 or more antibodies.

The kit may also comprise detection agents that specifically detect control proteins (e.g. positive control and/or negative control).

According to a particular embodiment, the kit comprises an antibody that specifically binds to fibronectin, an antibody that specifically binds to FAK and an antibody that specifically binds to MEK1. The number of target proteins for the antibodies of the kit is preferably no greater than 10, 15 or 20.

Additional contemplated antibodies for the kit include those that specifically bind to f3-Actin, C-Raf, N-Cadherin and/or an antibody that specifically binds to P90RSK_pT573.

It will be appreciated that additional kit components are contemplated that are useful for detecting the determinants disclosed herein on the polynucleotide level. Such determinants include primers and/or probes that are capable of specifically hybridizing to the determinants. Thus, in one embodiment, the kit comprises probes or primers that are capable of hybridizing to fibronectin, FAK and MEK1. The kit preferably comprises pimers/probes that hybridize to no more than 10, 15 or 20 target nucleic acids.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND METHODS

Plasma from breast cancer patients (ages 37-82) was collected. The criteria for inclusion in the study was an early stage BC, and candidacy for total tumor dissection. Blood for plasma samples (10 ml) was taken at the time of entry to the study (before the dissection surgery). For 27 patients, an additional blood sample was collected 24 weeks in average after surgery. Median follow up duration of the patients in this study is 114 weeks. Blood samples were also collected from healthy women (n=22, ages above 40), to serve as a control group. An independent set of blood samples to be used for validation purposes was obtained from the Sheba Medical Center tissue bank (including healthy age-matched women as controls). Blood was collected into EDTA tubes (0.02%), and centrifuged at 1,500 g for 15 minutes. The supernatant (plasma) was collected, aliquoted and kept at −80° C. as source for sEVs purification.

Isolation of sEVs and RPPA: To isolate sEVs from blood plasma, a reliable method was established using combination of size exclusion chromatography (SEC) and filtration. Plasma (2 ml) was first centrifuged at 300 g (10 min at 4° C.) followed by supernatant centrifugation at 10,000 g for 10 min. The 2 ml plasma was then concentrated to 0.5 ml using Nanosep Omega 300 kDa filters (PALL Life Science, Canada). Concentrated plasma samples were loaded on a qEV size chromatography column, separation size 70 nm (IZON, UK). The columns were washed with PBS, and 4 fractions of 1.5 ml were collected from the effluent. 100 ill of each fraction were used for particle counting by light scattering using the Nanosight instrument NS300 (Malvern Panalytical, UK), while the remainder of the samples were concentrated by repeated centrifugation through the 300 kDa filters (4 times, 10,000 rpm, 15 min each) to obtain a vesicle pellet. The concentrated vesicles on the filters were lysed in 50 ill RPPA lysis buffer (1% Triton X-100, 50 mM Hepes, pH 7.4, 150 mM NaCl, 1.5 mM MgCl2, 1 mM EGTA, 100 mM NaF, 10 mM Na pyrophosphate, 1 mM Na3VO4, 10% glycerol and protease inhibitors). Protein concentrations were measured by Bradford assay (BioRad). Protein lysates were analyzed by the Reverse Phase Protein Array (RPPA) core facility of the MD Anderson Cancer Center (Houston, Tex.). Results were normalized for protein loading as follows: the median for each antibody across all samples was calculated, and the results were median-centered for each antibody. Then the medians of each sample across all antibodies were measured. Samples with extremely low or high medians were considered to be outliers with either very low or high protein content and were removed from further analysis.

Statistical analysis: Statistical analysis of the RPPA results was performed with R, using the following packages. Determination of differentially expressed proteins between pre-surgery patients, post-surgery samples and healthy controls was performed using the LIMMA package. Comparisons between post-surgery and pre-surgery samples per patient were analyzed by paired t-test. K-Nearest neighbors (kNN) tests were performed using the Caret package, and optimized by manipulating several parameters including the number of neighbors and the number of proteins. Validation was done by the leave-one-out cross validation method. Elastic net regression was performed using the glmnet and caret packages. Receiver operating characteristic (ROC) curves were generated by the plotROC package in R and by the easyROC tool (www(dot)biosoft(dot)hacettepe(dot)edu(dot)tr/easyROC/). Area under the curve (AUC), Correlations were performed using the Hmisc package. Hierarchical clustering of the data was performed using the gplots package. Partition clustering was visualized by the Factoextra package. Decision trees and random forest models were built using the rpart and ranger packages, respectively. P-values less than 0.05 were considered statistically significant.

Immunoblotting: Total proteins were extracted from EVs using a lysis buffer containing 0.2% Triton-X-100, 50 mM Hepes pH 7.5, 100 mM NaCl, 1 mM MgCl2, 50 mM NaF, 0.5 mM Na3VO4, 20 mM β-glycerophosphate, 1 mM phenylmethylsulphonyl fluoride, 10m/mlleupeptin and 10m/m1 aprotinin. EVs lysates were centrifuged at 14,000 rpm for 15 min at 4° C., protein concentration of the supernatants was measured by Bradford assay (Bio-Rad, Hercules, Calif.). Equal amounts of proteins were analyzed by SDS—polyacrylamide gel electrophoresis and Western Blotting (WB) using the indicated antibodies. Equal volumes of lysates were analyzed using the Coomassie dye Imperial Protein Stain (Thermo Fisher). Antibodies used in this study: TSG101 (ABCAM, ab30871), HSC70 (Enzo Life Sciences, ADI-SPA-822), ALIX (Santa Cruz, SC-53540), FAK (Santa Cruz, SC-932), MEK1 (Cell Signaling Technology, #9124), Fibronectin (DSHB, University of Iowa).

Transmission electronic microscopy: Isolated extracellular vesicles (3 μL) were applied to glow-discharged, 300 mesh formvar/carbon coated copper TEM grids (Electron Microscopy Sciences) for 30 seconds. Excess liquid was blotted, followed by washing with distilled water and staining with 2% uranyl acetate. Samples were visualized in an FEI Tecnai T12 TEM operated at 120 kV, equipped with a TVIPS TemCam-XF416.

### RESULTS

Isolation of sEVs from Human Blood Plasma

The sEVs proteome has been proposed to provide useful clinical information for detection and stratification of BC (6). Thus, an efficient, robust and reliable method for sEVs isolation is a critical need (12). A major challenge of sEVs isolation from plasma is to avoid contamination of abundant plasma proteins such as albumin while concurrently collecting sufficient sEV proteins for global proteomic or clinical analysis. To simultaneously accomplish these two requirements, we established an efficient protocol that requires only 2 ml plasma, and results in high yields of purified sEVs. The isolation protocol is illustrated in FIG. 1A, and includes two important steps, filtration and size exclusion chromatography (SEC). SEC is considered to be a better method for diagnostic assays compared to standard ultracentrifugation as it retains sEVs integrity (13,14) and concomitantly decreases plasma protein contaminants (15). Following protocol calibration, the SEC eluent was fractionated into 4 fractions of 1.5 ml each and particle numbers were measured by light scattering (NanoSight). As shown, the third and fourth fractions had the highest numbers of particles with average size of ˜110 nm diameter (FIG. 1B), a characteristic size of sEVs (5). Both fractions consist typical exosomal-like markers, including TSG101, ALIX and HSP70 (FIG. 1C). The abundance of albumin in the isolated fractions compared to total plasma was assessed by Coomassie Blue staining of similar lysate volumes, and showed high albumin levels in the fourth fraction (FIG. 1D). Fraction purity was calculated as log ratio between particle number and protein concentration in each fraction (12), with highest purity in fraction number 3 (FIG. 1E). Accordingly, fraction 3 was used for further analysis and proteomic profiling.

Proteomic Analysis and Diagnostic Signature

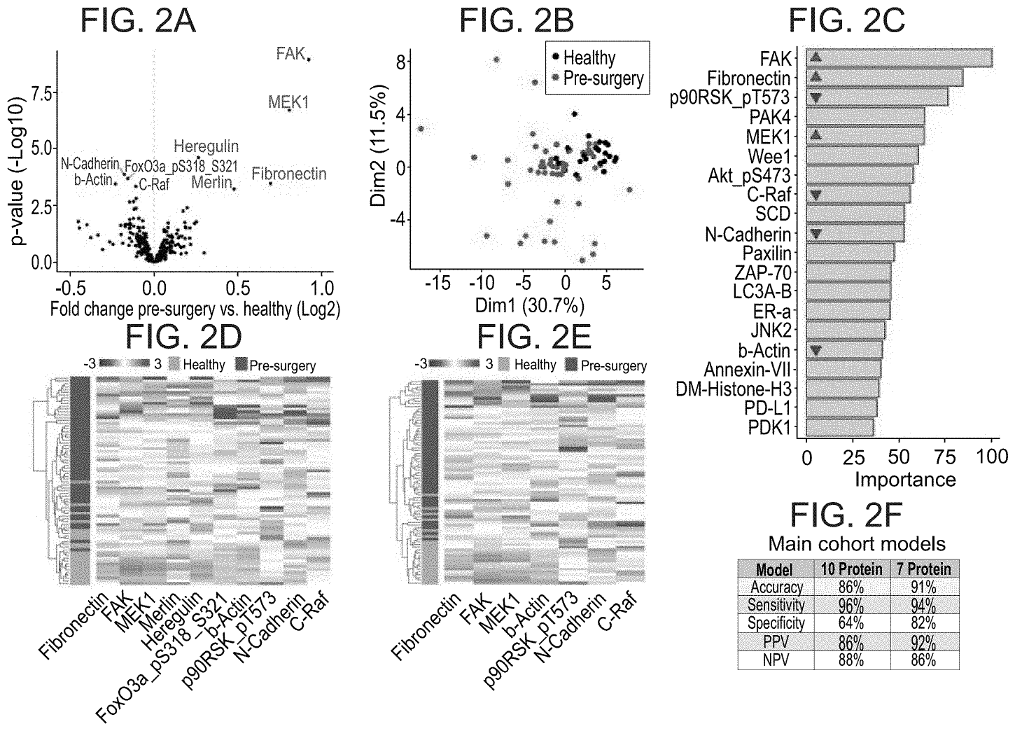

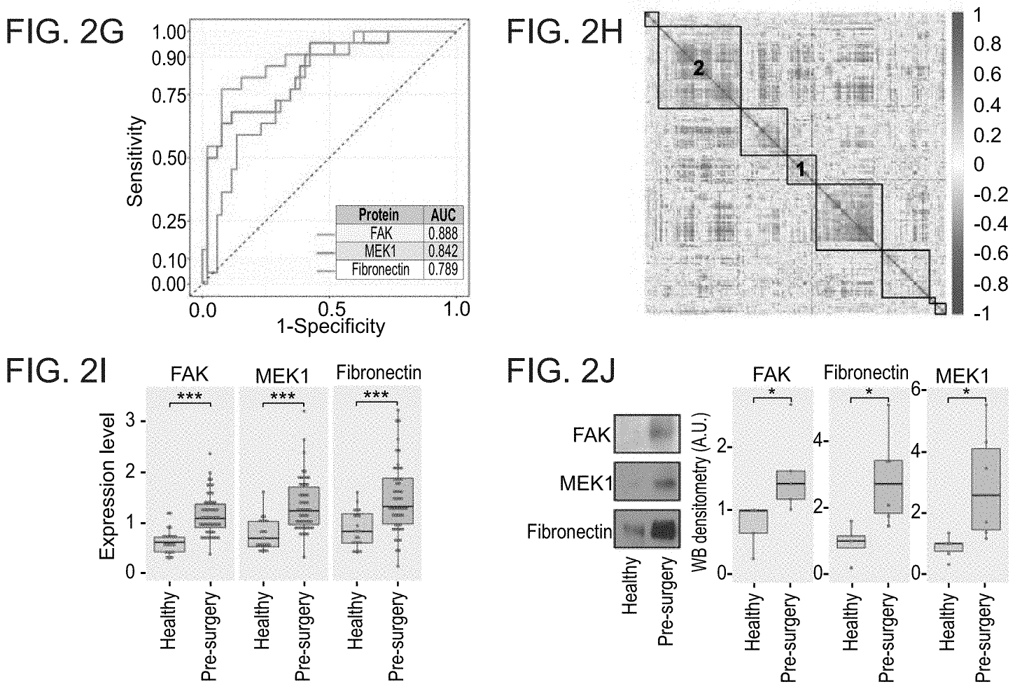

Protein extracts from the sEVs-enriched fractions (50 μg) were analyzed by RPPA (Reverse Phase Protein Array, core facility of MDACC, Houston, Tex.) to assess total and phosphoprotein levels of ˜276 cellular proteins that primarily associated with cancer-related signaling pathways (16). The results of significantly differentially expressed proteins in pre-surgery BC samples compared to healthy controls are summarized in the volcano plot shown in FIG. 2A, and most prominent proteins are indicated. Among the upregulated proteins, FAK, MEK1 and Fibronectin were highly enriched in EVs driven from plasma of BC patients, consistent with previous reports on FAK (17) and fibronectin in EVs (18).

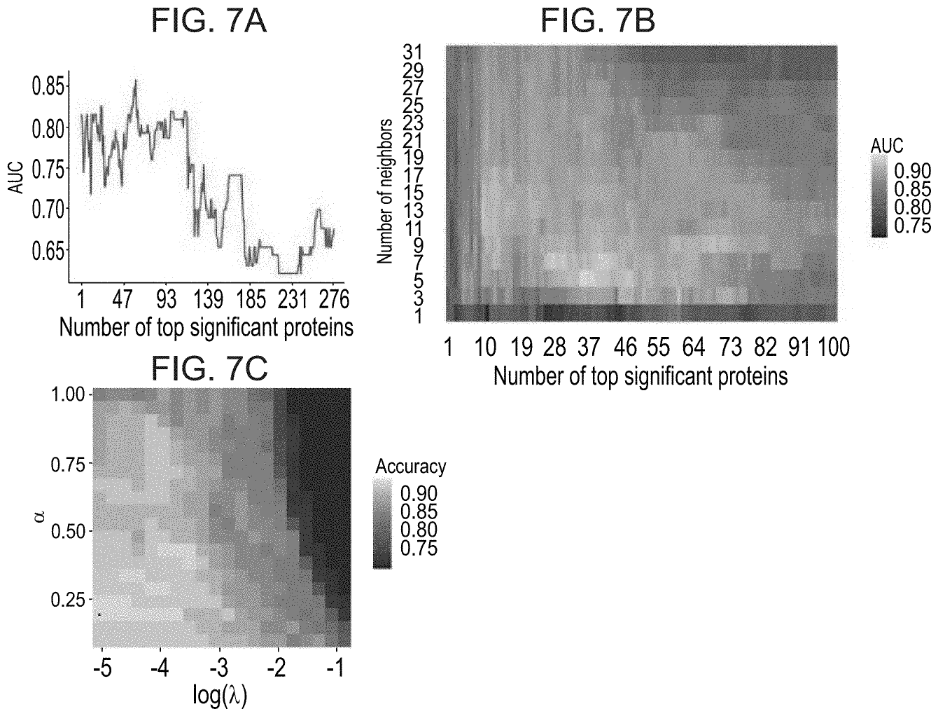

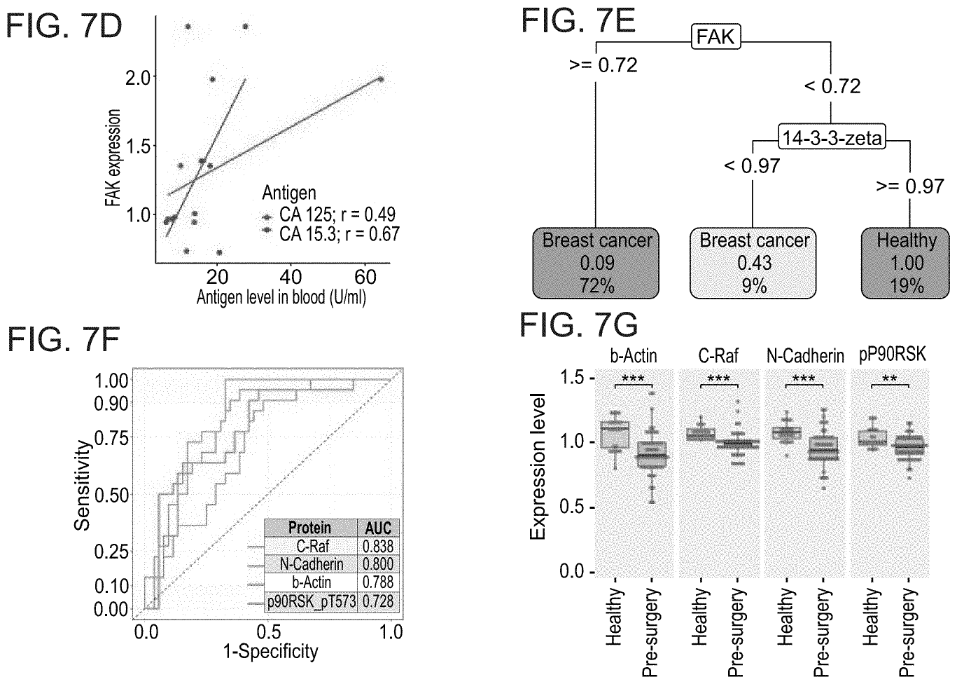

To generate a protein signature that stratify breast cancer patients and healthy controls, we performed k-nearest neighbor test, a robust method for predicting outcomes based on array data (19). We used leave-one-out cross validation and ROC-AUC as the performance metric (19). Using 1 neighbor (k=1) and performing the test with the N top significant differently-expressed proteins (with N starting from 1, being FAK, up to 276, the number of proteins in the array, ordered by increasing p-value), we discovered that the best partition is with N=60 proteins (FIG. 7A, FIG. 2B). To generate a signature composed of a small number of proteins that will maximize the classification, we performed kNN models for k=1..31 and N=1..100 (FIG. 7B). We discovered a local maximum point for AUC at N=10 (FIG. 7B), suggesting that these 10 proteins, of which were upregulated and 5 downregulated in BC patients, provide a good classifying signature of BC versus healthy women. Unsupervised clustering of the entire cohort of patients and controls using these 10 proteins (FIG. 2C) indeed showed a good separation between BC patients and healthy controls. The clustering yielded high sensitivity (true positive rate) of 96%, with a specificity (true negative rate) of 64%. Positive and negative predictive values of the signature are 86% and 88%, respectively (FIG. 2F).

In order to improve the signature classification accuracy, in particular to increase specificity, we applied another classification method on the main cohort of 52 patients and 22 healthy controls—logistic regression with elastic net penalty. To obtain the best accuracy, the model was trained on the cohort (using 10-fold cross validation) to tune the parameters λ (which controls the total extent of the penalty) and a (which controls the shift between L1 [lasso] and L2 [ridge] penalties) (FIG. 7C). The best accuracy was 92.3%, similar to the kNN model. The most influential predictors in the model (in term of their coefficient p-values) are given in FIG. 2D. Seven out of ten proteins in the kNN signature discovered above are among the most influential predictors of the elastic net model. We, therefore, clustered the cohort using these 7 proteins (FIG. 1E), achieving better prediction accuracy, with improved specificity of 82% (FIG. 2F). Interestingly, by using a similarity matrix, showing the correlation between any two proteins in the BC cohort (FIG. 2I), we found that all the 3 upregulated proteins (FAK, MEK1 and Fibronectin) belong to the same cluster (cluster #1), indicating that each of these proteins is correlated with the others. Among the downregulated proteins in the signature, 3 of them (β-Actin, C-Raf, and N-Cadherin) also belong to the same cluster (cluster #2 in the figure).

To assess the prediction accuracy of each individual protein among the seven proteins in the signature, we performed a Receiver Operating Characteristic (ROC) curve analysis on the upregulated proteins in the signature (FIG. 2G). We took the area under the ROC curve (ROC-AUC) as a measurement of the predictive value of each protein. FAK, MEK1 and Fibronectin were found to have high AUC and high fold change between BC patients and healthy controls (FIG. 2H). We observed a positive correlation between FAK expression in the plasma of the BC patients and the plasma levels of CA 125 and CA 15-3, two commonly used circulating markers for BC (FIG. 7D), further demonstrating the clinical relevance of our identified markers. The strong predictive value of FAK was also shown by building a decision tree model using all available predictors. FAK was found to be the protein that classified the cohort with the highest accuracy (FIG. 7E). To validate the RPPA results, the levels of the upregulated proteins in the signature (FAK, MEK1 and fibronectin) were also found to be increased in BC compared to healthy samples using Western blotting (FIG. 2J). ROC-AUC analysis was also performed on the 4 downregulated proteins in the signature and AUC of 0.728-0.838 were obtained (FIG. 7F, G).

Validating the Predicative Power of the Signature

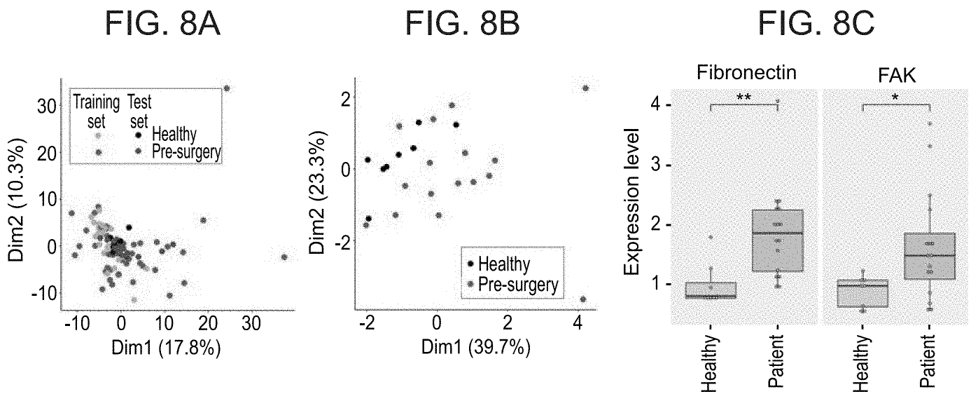

To validate the predictive strength of our analysis, we obtained an independent test set of plasma samples from other resources in the Sheba Medical Center. This set included plasma samples taken from 16 breast cancer patients obtained during surgery, and 8 control samples from healthy women. There was no apparent batch effect in the RPPA results between the main cohort and the test set (FIG. 8A). Using the identified signature of 7 proteins shown in FIG. 2E, we could cluster the breast cancer patients and healthy controls with high accuracy of 88% (FIG. 3A, 8B). For the individual predictors, we could obtain high ROC-AUCs for FAK and fibronectin in the test set (FIG. 3B, 8C), thus validating their predictive value and highlight the power of our prediction approach.

To determine whether we can increase the test set prediction accuracy, we tested several other classification models (20), and compared the results to the classification by our 7 proteins signature. Out of the 276 proteins examined in the main cohort RPPA, 267 were also examined in the test set RPPA. As input, the models were given the expression levels of these 267 proteins. Each model was trained on the main cohort using different cross validation methods, and then tested on the independent test set. As shown in FIG. 3C, although random forest achieved better sensitivity (true positive rate), overall our signature had the best specificity (true negative rate), and thus overall accuracy.

Biomarkers for Detection of Cancer Stage

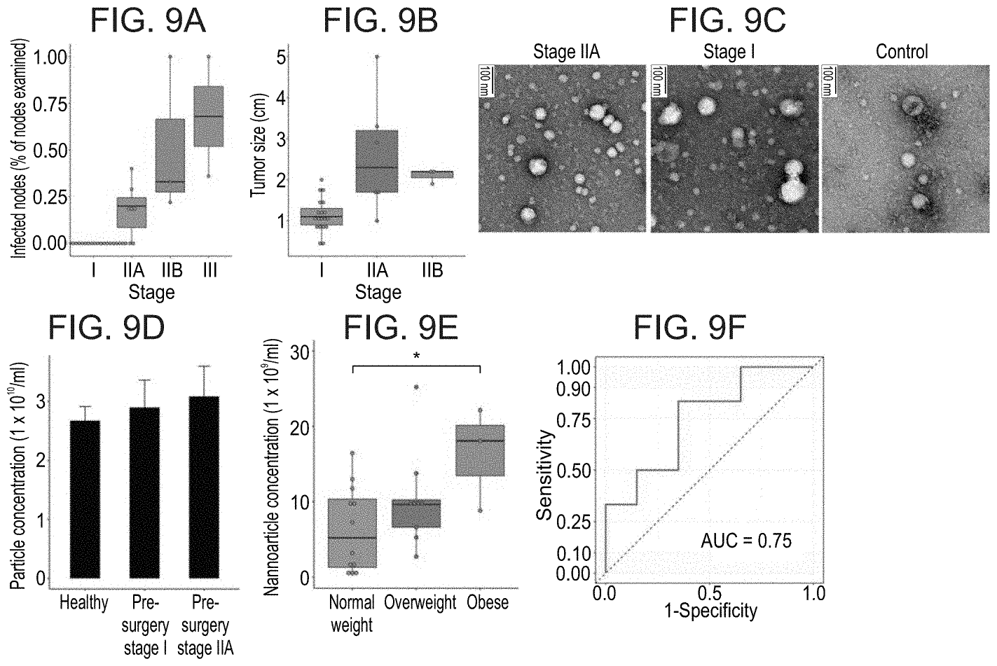

Next, we examined significant differences between breast cancer stages. As particle size distributions revealed no significant difference between pre-surgery patients and controls, we divided the pre-surgery patients into two categories based on tumor stage, as classified by the TNM (Tumor size, Nodes infected and Metastasis) system (FIG. 9A,B). Our cohort was composed mainly of stage I and IIA patients. While particle size distribution of stage I BC patients was not different from healthy controls, stage IIA particles exhibited a shift toward lower particles size, as measured by side scattering analysis (FIG. 4A). This difference in size distribution was also observed by transmission electronic microscopy analysis (FIG. 9C). Moreover, the number of small EVs (size <100 nm), which might include exosomes or exosomes-like vesicles (ELV), was significantly increased in stage IIA patients (FIG. 4B) compared to stage I or healthy samples, although total number of particles remained similar in the three groups (FIG. 9D). ROC analysis of sEV (<100 nm) concentration in the plasma of stage II BC versus healthy samples yielded an AUC of 0.75 (FIG. 9F). Interestingly, an increased number of sEV of less than 100 nm was also associated with increase in Body Mass Index (BMI) (FIG. 9E), but not with any other clinical parameter.

Next, we looked for sEVs proteins that can distinguish between Stage I, Stage IIA and healthy samples. To that end, we first built a protein signature unique for each stage using kNN, as well as a signature to differentiate stage IIA from stage I patients (FIG. 10A, 10B). Using patient versus healthy signatures, we could cluster stage I (FIG. 4C) and stage IIA (FIG. 4D) patients apart from healthy controls with high accuracy. Logistic regression done for each stage versus healthy samples, corroborated most of the proteins in each kNN signature (FIG. 4C, D right panel). While the protein signatures for stage I and stage IIA shared several proteins, including the 3 most prominent FAK, MEK1 and fibronectin, each also had unique proteins (FIG. 10C), which were analyzed by ROC-AUC to find unique markers for each stage. The best protein markers when comparing stage I patients (FIG. 10D) or stage IIA patients (FIG. 10E) to healthy controls are EGFR and P-cadherin, respectively. Further, the signature with the best AUC that differentiate between stage IIA and stage I consists of 2 proteins, including IGFRβ which has the best ROC-AUC (FIG. 10F). The expression level of markers in our cohort is given in FIG. 10H. Several of the prominent markers for stage IIA (including P-cadherin and TAZ) were decreased in plasma sEVs of BC patients compared to control (FIG. 10H). In light of the increased number of smaller EVs (<100 nm) in plasma of stage IIA patients (FIG. 4B), TAZ and P-cadherin are indeed significantly negatively correlated to smaller EV number (FIG. 10G). This suggests that both the number of smaller EV and the expression levels of specific proteins can help to distinguish stage IIA from stage I patients.

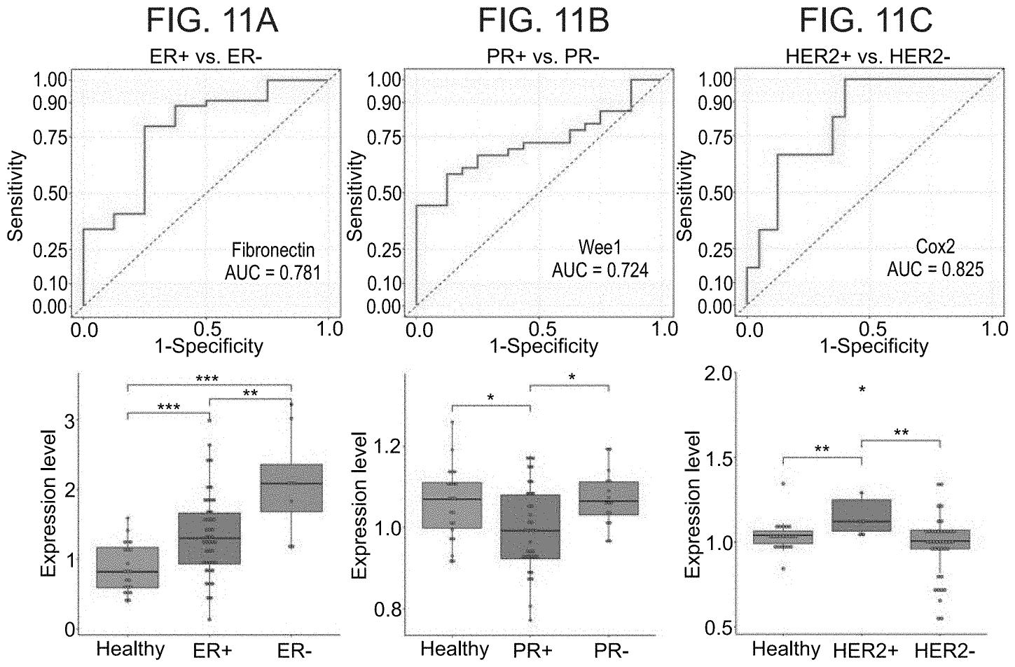

Using similar methods, we could also find characteristic proteins for different BC subtypes (ER+, PR+, HER2+). Fibronectin was found by ROC analysis to differentiate between ER positive and negative, Wee 1 between PR positive and negative and Cox2 between HER2 positive and negative (FIG. 11A-C). Consistent with our findings, Cox2 was previously shown to correlate to HER2 status in BC (21).

Protein Signature for Risk of Relapse

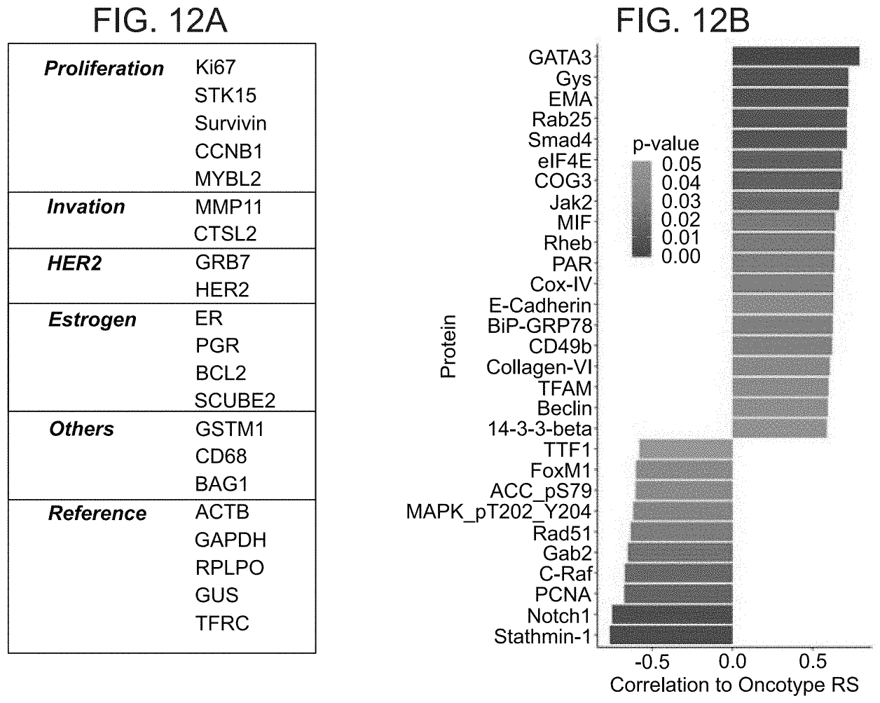

Within the time frame of the study, four patients underwent relapse, and three out of these four were analyzed by RPPA (the forth was a technical outlier in the RPPA). To examine if these relapsed patients belong to a discrete group, we used partition clustering analysis of pre-surgery samples. As shown in FIG. 5A, the pre-surgery samples are significantly variant, consisting of a few distinct clusters, including a unique cluster of patients with a risk of relapse (cluster 4 in FIG. 5A). This segregation suggests that a protein signature can potentially be built to predict relapse using our methodology of sEV extraction and RPPA analysis. To explore this possibility, we obtained the Oncotype recurrence score (RS) of 16 patients from our cohort (FIG. 5B). The Oncotype RS is an excellent clinical test to estimate the likelihood of relapse and the benefit of chemotherapy in ER-positive BC patients based on the RNA expression levels of 21 selected genes (22) (FIG. 12A). High Oncotype scores (>25) are considered to predict a high risk of relapse. Importantly, two of the patients (No. 14 and 16, FIG. 5B) with a score above 25 indeed relapsed within the duration of our study. One of them, patient No. 14 (RS=28), had RPPA data and was clustered in the relapse-risk cluster (cluster 4, FIG. 5A). An additional patient with high oncotype score (RS=31, patient No. 15, FIG. 5B) was also found to be included in the high relapse potential cluster in our partitioning analysis (FIG. 5A), thus supporting a high likelihood of recurrence in patients of this cluster. Interestingly, we observed an increase in the RS values along the major principle component (FIG. 5A), implying that combining the proteomic analysis described here together with RS scoring could substantially improve prediction potential.

To gain better insight on protein expression patterns of relapsed patients, we compared the RPPA data of the three relapsed patients plus patient no. 15 (FIG. 5B), who is part of cluster 4 (FIG. 5A) and has high RS, to the RPPA data from the other patients. Although many differentially expressed proteins (FIG. 5C) were observed, the highest and most significant was HSP70, which was previously associated with tumor recurrence (23). Furthermore, we found good correlations between the expression of several proteins to the Oncotype score (FIG. 12B), suggesting that such proteins can be used to predict relapse in patients that did not undergo Oncotype score evaluation. Together, our findings demonstrate the power of sEVs proteome to predict recurrence risk, and highlighting its clinical potential pending additional validation studies.

Analysis of EVs Post-Surgery

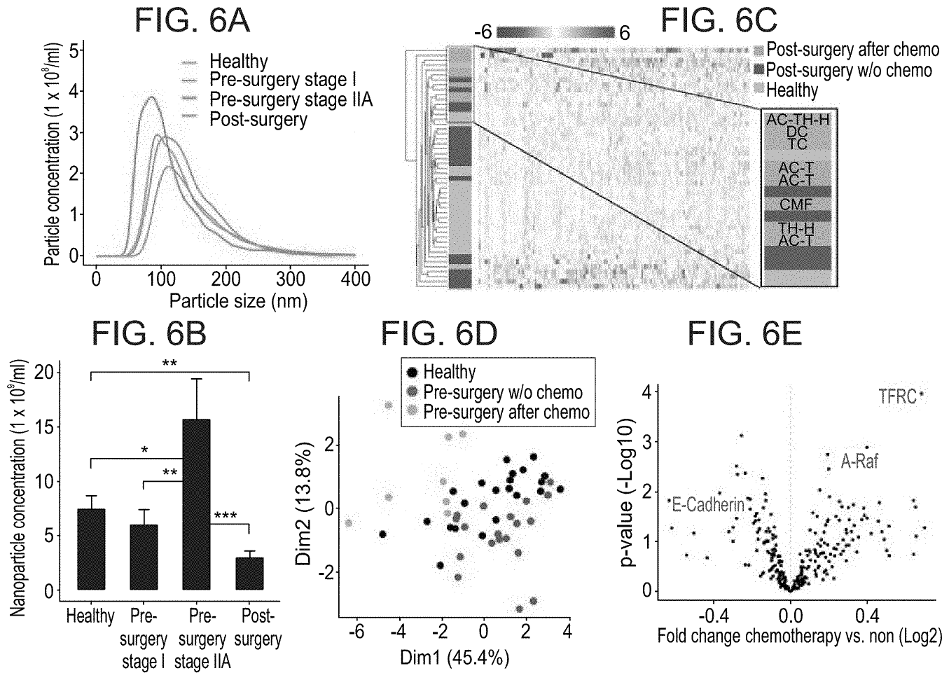

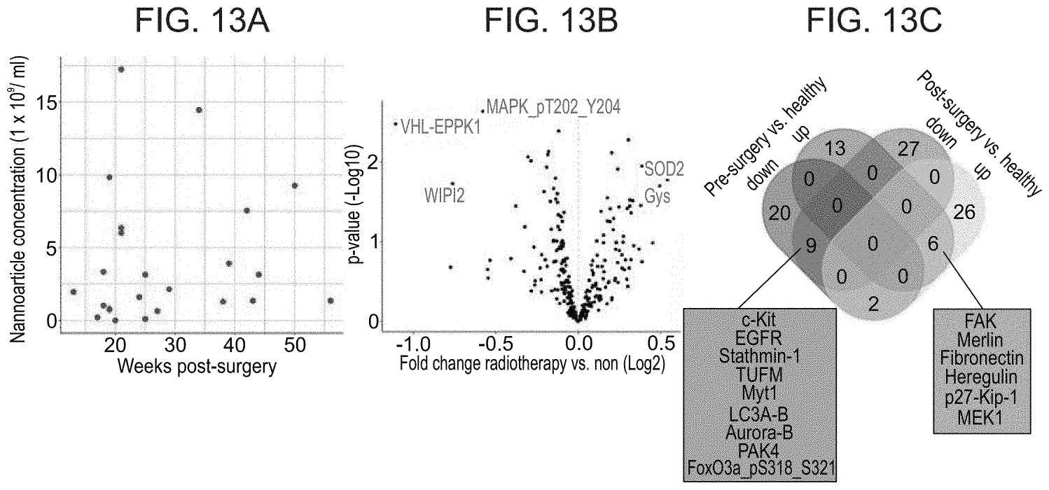

Next, we analyzed EVs of 27 patients post-surgery. Plasma samples were collected in average 24 weeks after surgery. Patients undergoing chemotherapy were sampled during or after chemotherapy, and in most cases before any other treatment. Other patients were sampled before or during radiotherapy and/or hormone therapy. sEVs were isolated as described in FIG. 1A. Light scattering analysis showed a significant shift in particle concentration histogram toward larger particle sizes of ˜150 nm diameter (FIG. 6A), concurrent with a significant reduced number of sEVs in the smaller range (<100 nm), not only compared to the pre-surgery patients but also compared to healthy controls (FIG. 6B). Importantly, these effects were not correlated to the time of plasma collection after surgery (FIG. 13A), but most likely were due to the applied therapy. Indeed, unsupervised clustering separated healthy samples from the post-surgery samples (FIG. 6C). Further, the clustering separated to some degree patients who received chemotherapy compared to those who didn't (FIG. 6C, chemotherapy regiment is indicated). Principal component analysis using all significantly different proteins between chemo-treated and non-treated patients (p-value <0.01) also reveals this separation between the 3 groups (FIG. 6D). Taken together, this suggests that treatment induces substantial difference in EVs content in post-surgery patients, mainly due to the chemotherapy treatment. Specifically, EVs from patients that underwent chemotherapy were enriched in metastasis promoting factors such as transferrin receptor (TFRC) (24), concurrent with a substantial downregulation of E-cadherin, suggesting that tumors may undergo EMT on therapy, as expected (25) (FIG. 6E).

Similarly, we analyzed proteins affected by radiotherapy (FIG. 13B; 14 patients received radiotherapy versus 13 did not) and identified superoxide dismutase 2 (SOD2) as the highest upregulated protein possibly as a result of reactive oxygen species (ROS) generation and oxidative stress induced by radiation (26,27). Finally, we compared the differentially expressed proteins in pre-surgery and post-surgery versus healthy samples and observed a few proteins, including the biomarkers for BC identified above (FAK, MEK1 and fibronectin) that remained upregulated in EVs of post-surgery samples (FIG. 13C). By a pairwise comparison of post- and pre-surgery samples for the same patients, we were able to generate a list of proteins that change following the surgery and treatment.

### REFERENCE

1. Seely, J. M., and Alhassan, T. (2018) Screening for breast cancer in 2018-what should we be doing today? Current oncology 25, S115-S124
2. Jia, Y., Chen, Y., Wang, Q., Jayasinghe, U., Luo, X., Wei, Q., Wang, J., Xiong, H., Chen, C., Xu, B., Hu, W., Wang, L., Zhao, W., and Zhou, J. (2017) Exosome: emerging biomarker in breast cancer. Oncotarget 8, 41717-41733
3. Li, A., Zhang, T., Zheng, M., Liu, Y., and Chen, Z. (2017) Exosomal proteins as potential markers of tumor diagnosis. J Hematol Oncol 10, 175
4. Hessvik, N. P., and Llorente, A. (2018) Current knowledge on exosome biogenesis and release. Cellular and molecular life sciences: CMLS 75, 193-208
5. Durcin, M., Fleury, A., Taillebois, E., Hilairet, G., Krupova, Z., Henry, C., Truchet, S., Trotzmuller, M., Kofeler, H., Mabilleau, G., Hue, 0., Andriantsitohaina, R., Martin, P., and Le Lay, S. (2017) Characterisation of adipocyte-derived extracellular vesicle subtypes identifies distinct protein and lipid signatures for large and small extracellular vesicles. J Extracell Vesicles 6, 1305677
6. Rontogianni, S., Synadaki, E., Li, B., Liefaard, M. C., Lips, E. H., Wesseling, J., Wu, W., and Altelaar, M. (2019) Proteomic profiling of extracellular vesicles allows for human breast cancer subtyping. Communications biology 2, 325
7. Chen, G., Huang, A. C., Zhang, W., Zhang, G., Wu, M., Xu, W., Yu, Z., Yang, J., Wang, B., Sun, H., Xia, H., Man, Q., Zhong, W., Antelo, L. F., Wu, B., Xiong, X., Liu, X., Guan, L., Li, T., Liu, S., Yang, R., Lu, Y., Dong, L., McGettigan, S., Somasundaram, R., Radhakrishnan, R., Mills, G., Lu, Y., Kim, J., Chen, Y. H., Dong, H., Zhao, Y., Karakousis, G. C., Mitchell, T. C., Schuchter, L. M., Herlyn, M., Wherry, E. J., Xu, X., and Guo, W. (2018) Exosomal PD-Ll contributes to immunosuppression and is associated with anti-PD-1 response. Nature 560, 382-386
8. Xu, R., Rai, A., Chen, M., Suwakulsiri, W., Greening, D. W., and Simpson, R. J. (2018) Extracellular vesicles in cancer—implications for future improvements in cancer care. Nature reviews. Clinical oncology 15, 617-638
9. Boellner, S., and Becker, K. F. (2015) Reverse Phase Protein Arrays-Quantitative Assessment of Multiple Biomarkers in Biopsies for Clinical Use. Microarrays 4, 98-114
10. Lu, Y., Ling, S., Hegde, A. M., Byers, L. A., Coombes, K., Mills, G. B., and Akbani, R. (2016) Using reverse-phase protein arrays as pharmacodynamic assays for functional proteomics, biomarker discovery, and drug development in cancer. Seminars in oncology 43, 476-483
11. Mertins, P., Yang, F., Liu, T., Mani, D. R., Petyuk, V. A., Gillette, M. A., Clauser, K. R., Qiao, J. W., Gritsenko, M. A., Moore, R. J., Levine, D. A., Townsend, R., Erdmann-Gilmore, P., Snider, J. E., Davies, S. R., Ruggles, K. V., Fenyo, D., Kitchens, R. T., Li, S., Olvera, N., Dao, F., Rodriguez, H., Chan, D. W., Liebler, D., White, F., Rodland, K. D., Mills, G. B., Smith, R. D., Paulovich, A. G., Ellis, M., and Carr, S. A. (2014) Ischemia in tumors induces early and sustained phosphorylation changes in stress kinase pathways but does not affect global protein levels. Molecular & cellular proteomics: MCP 13, 1690-1704
12. Tang, Y. T., Huang, Y. Y., Zheng, L., Qin, S. H., Xu, X. P., An, T. X., Xu, Y., Wu, Y. S., Hu, X. M., Ping, B. H., and Wang, Q. (2017) Comparison of isolation methods of exosomes and exosomal RNA from cell culture medium and serum. Int J Mol Med 40, 834-844
13. Hong, C. S., Funk, S., Muller, L., Boyiadzis, M., and Whiteside, T. L. (2016) Isolation of biologically active and morphologically intact exosomes from plasma of patients with cancer. J Extracell Vesicles 5, 29289
14. Stranska, R., Gysbrechts, L., Wouters, J., Vermeersch, P., Bloch, K., Dierickx, D., Andrei, G., and Snoeck, R. (2018) Comparison of membrane affinity-based method with size-exclusion chromatography for isolation of exosome-like vesicles from human plasma. Journal of translational medicine 16, 1
15. Wang, T., Anderson, K. W., and Turko, I. V. (2017) Assessment of Extracellular Vesicles Purity Using Proteomic Standards. Anal Chem 89, 11070-11075
16. Grote, T., Siwak, D. R., Fritsche, H. A., Joy, C., Mills, G. B., Simeone, D., Whitcomb, D. C., and Logsdon, C. D. (2008) Validation of reverse phase protein array for practical screening of potential biomarkers in serum and plasma: accurate detection of CA19-9 levels in pancreatic cancer. Proteomics 8, 3051-3060
17. Galindo-Hernandez, 0., Villegas-Comonfort, S., Candanedo, F., Gonzalez-Vazquez, M. C., Chavez-Ocana, S., Jimenez-Villanueva, X., Sierra-Martinez, M., and Salazar, E. P. (2013) Elevated concentration of microvesicles isolated from peripheral blood in breast cancer patients. Arch Med Res 44, 208-214
18. Moon, P. G., Lee, J. E., Cho, Y. E., Lee, S. J., Chae, Y. S., Jung, J. H., Kim, I. S., Park, H. Y., and Baek, M. C. (2016) Fibronectin on circulating extracellular vesicles as a liquid biopsy to detect breast cancer. Oncotarget 7, 40189-40199
19. Parry, R. M., Jones, W., Stokes, T. H., Phan, J. H., Moffitt, R. A., Fang, H., Shi, L., Oberthuer, A., Fischer, M., Tong, W., and Wang, M. D. (2010) k-Nearest neighbor models for microarray gene expression analysis and clinical outcome prediction. The pharmacogenomics journal 10, 292-309
20. Ganggayah, M. D., Taib, N. A., Har, Y. C., Lio, P., and Dhillon, S. K. (2019) Predicting factors for survival of breast cancer patients using machine learning techniques. BMC medical informatics and decision making 19, 48
21. Jana, D., Sarkar, D. K., Ganguly, S., Saha, S., Sa, G., Manna, A. K., Banerjee, A., and Mandal, S. (2014) Role of Cyclooxygenase 2 (COX-2) in Prognosis of Breast Cancer. Indian journal of surgical oncology 5, 59-65
22. Paik, S., Shak, S., Tang, G., Kim, C., Baker, J., Cronin, M., Baehner, F. L., Walker, M. G., Watson, D., Park, T., Hiller, W., Fisher, E. R., Wickerham, D. L., Bryant, J., and Wolmark, N. (2004) A multigene assay to predict recurrence of tamoxifen-treated, node-negative breast cancer. The New England journal of medicine 351, 2817-2826
23. Rothammer, A., Sage, E. K., Werner, C., Combs, S. E., and Multhoff, G. (2019) Increased heat shock protein 70 (Hsp70) serum levels and low NK cell counts after radiotherapy-potential markers for predicting breast cancer recurrence? Radiation oncology 14, 78
24. Shen, Y., Li, X., Dong, D., Zhang, B., Xue, Y., and Shang, P. (2018) Transferrin receptor 1 in cancer: a new sight for cancer therapy. American journal of cancer research 8, 916-931
25. Liu, F., Gu, L. N., Shan, B. E., Geng, C. Z., and Sang, M. X. (2016) Biomarkers for EMT and MET in breast cancer: An update. Oncol Lett 12, 4869-4876
26. Kim, W., Lee, S., Seo, D., Kim, D., Kim, K., Kim, E., Kang, J., Seong, K. M., Youn, H., and Youn, B. (2019) Cellular Stress Responses in Radiotherapy. Cells 8
27. Zhang, Z., Lang, J., Cao, Z., Li, R., Wang, X., and Wang, W. (2017) Radiation-induced SOD2 overexpression sensitizes colorectal cancer to radiation while protecting normal tissue. Oncotarget 8, 7791-7800
28. Luo, M., and Guan, J. L. (2010) Focal adhesion kinase: a prominent determinant in breast cancer initiation, progression and metastasis. Cancer Lett 289, 127-139
29. Lee, J., Lim, B., Pearson, T., Choi, K., Fuson, J. A., Bartholomeusz, C., Paradiso, L. J., Myers, T., Tripathy, D., and Ueno, N. T. (2019) Anti-tumor and anti-metastasis efficacy of E6201, a MEK1 inhibitor, in preclinical models of triple-negative breast cancer. Breast cancer research and treatment 175, 339-351
30. Nowsheen, S., Aziz, K., Panayiotidis, M. I., and Georgakilas, A. G. (2012) Molecular markers for cancer prognosis and treatment: have we struck gold? Cancer Lett 327, 142-152
31. Paredes, J., Correia, A. L., Ribeiro, A. S., Albergaria, A., Milanezi, F., and Schmitt, F. C. (2007) P-cadherin expression in breast cancer: a review. Breast Cancer Res 9, 214
32. Zhou, X., and Lei, Q. Y. (2016) Regulation of TAZ in cancer. Protein & cell 7, 548-561
33. M. A. Panteleev, A. A. A., A. N. Balandina, A. V. Belyaev, D. Y. Nechipurenko, S. I. Obydennyi, A. N. Sveshnikova, A. M. Shibeko, F. I. Ataullakhanov. (2017) Extracellular vesicles of blood plasma: content, origin, and properties. Biochem. Moscow Suppl. Ser. A 11, 187-192
34. Whiteside, T. L. (2018) The emerging role of plasma exosomes in diagnosis, prognosis and therapies of patients with cancer. Contemporary oncology 22, 38-40
35. Sparano, J. A., Gray, R. J., Makower, D. F., Pritchard, K. I., Albain, K. S., Hayes, D. F., Geyer, C. E., Jr., Dees, E. C., Goetz, M. P., Olson, J. A., Jr., Lively, T., Badve, S. S., Saphner, T. J., Wagner, L. I., Whelan, T. J., Ellis, M. J., Paik, S., Wood, W. C., Ravdin, P. M., Keane, M. M., Gomez Moreno, H. L., Reddy, P. S., Goggins, T. F., Mayer, I. A., Brufsky, A. M., Toppmeyer, D. L., Kaklamani, V. G., Berenberg, J. L., Abrams, J., and Sledge, G. W., Jr. (2018) Adjuvant Chemotherapy Guided by a 21-Gene Expression Assay in Breast Cancer. The New England journal of medicine 379, 111-121
36. van Niel, G., D'Angelo, G., and Raposo, G. (2018) Shedding light on the cell biology of extracellular vesicles. Nat Rev Mol Cell Biol 19, 213-228
37. Keklikoglou, I., Cianciaruso, C., Guc, E., Squadrito, M. L., Spring, L. M., Tazzyman, S., Lambein, L., Poissonnier, A., Ferraro, G. B., Baer, C., Cassara, A., Guichard, A., Iruela-Arispe, M. L., Lewis, C. E., Coussens, L. M., Bardia, A., Jain, R. K., Pollard, J. W., and De Palma, M. (2019) Chemotherapy elicits pro-metastatic extracellular vesicles in breast cancer models. Nature cell biology 21, 190-202
38. Wang, H. X., and Gires, 0. (2019) Tumor-derived extracellular vesicles in breast cancer: From bench to bedside. Cancer Lett 460, 54-64
39. Ludwik, K. A., Campbell, J. P., Li, M., Li, Y., Sandusky, Z. M., Pasic, L., Sowder, M. E., Brenin, D. R., Pietenpol, J. A., O'Doherty, G. A., and Lannigan, D. A. (2016) Development of a RSK Inhibitor as a Novel Therapy for Triple-Negative Breast Cancer. Molecular cancer therapeutics 15, 2598-2608

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating breast cancer of a subject in need thereof, the method comprising:
(a) isolating extracellular vesicles from serum and/or plasma of the subject to generate an isolated population of extracellular vesicles;
(b) lysing said isolated population of extracellular vesicles to generate a composition comprising components of said extracellular vesicles;
(c) measuring the amount of MEK1 in said composition; and
(d) diagnosing the subject with breast cancer when a level of said MEK1 in said composition is above a predetermined threshold; and upon confirmation of breast cancer in the subject
(e) administering to the subject a therapeutic agent which is directed against breast cancer cells of the subject.

2. The method of claim 1, further comprising measuring the amount of fibronectin and FAK in said composition, wherein a level of said MEK1, said fibronectin and said FAK in said composition above a predetermined threshold is indicative that the subject has breast cancer.

3. The method of claim 1, wherein said isolated population of extracellular vesicles are between 30-150 nM in diameter.

4. The method of claim 1, wherein said isolating comprises purifying said extracellular vesicles from serum albumin.

5. The method of claim 1, wherein said isolated population of extracellular vesicles comprise exosomes.

6. The method of claim 4, wherein said purifying is effected by performing size exclusion chromatography and/or filtration on said isolated population of extracellular vesicles.

7. The method of claim 4, wherein said purifying is effected by depleting said composition of serum albumin.

8. A method of treating breast cancer of a subject in need thereof, the method comprising:
(a) isolating extracellular vesicles from serum and/or plasma of the subject to generate an isolated population of extracellular vesicles;
(b) lysing said population of extracellular vesicles to generate a composition comprising components of said extracellular vesicles;
(c) measuring the amount of each of the proteins MEK1, fibronectin, FAK, β-Actin, C-Raf, N-Cadherin and P90RSK_pT573 in said composition; and
(d) diagnosing the subject with breast cancer based on the amount of each of said proteins; and upon confirmation of breast cancer in the subject
(e) administering to the subject a therapeutic agent which is directed against breast cancer cells of the subject.

9. The method of claim 8, wherein said isolated population of extracellular vesicles are between 30-150 nM in diameter.

10. The method of claim 8, wherein said isolating comprises purifying said extracellular vesicles from serum albumin.

11. The method of claim 8, wherein said isolated population of extracellular vesicles comprise exosomes.

12. The method of claim 10, wherein said purifying is effected by performing size exclusion chromatography and/or filtration on said isolated population of extracellular vesicles.

13. The method of claim 10, wherein said purifying is effected by depleting said composition of serum albumin.

14. A kit for diagnosing breast cancer, the kit comprising an antibody that specifically binds to fibronectin, an antibody that specifically binds to FAK and an antibody that specifically binds to MEK1, wherein the number of target proteins for the antibodies of the kit is no greater than 20.

15. The kit of claim 14, wherein the number of target proteins for the antibodies of the kit is no greater than 10.

16. The kit of claim 14, further comprising an antibody that specifically binds to f3-Actin, an antibody that specifically binds to C-Raf, an antibody that specifically binds to N-Cadherin and an antibody that specifically binds to P90RSK_pT573.

17. The kit of claim 14, wherein at least one of said antibody is attached to a solid support.

18. The kit of claim 14, wherein at least one of said antibody is attached to a detectable moiety.
